(19) United States
(12) Patent Application Publication
(10) Pub. No.: US 2003/0152924 A1
(43) Pub. Date: Aug. 14, 2003

(54) Strand displacement detection of target nucleic acid

(76) Inventors: Edwin F. Ullman, Atherton, CA (US); Ming Wu, Castro Valley, CA (US)

Correspondence Address: Hana Verny, PETERS, VERNY, JONES & BIKSA LLP, Suite 6, 385 Sherman Avenue, Palo Alto, CA, 94306, US

(21) Appl. No.: 10/012,742

(22) Filed: Dec. 7, 2001

Related U.S. Application Data
(60) Provisional application No. 60/256,737, filed on Dec. 19, 2000.

(51) Int. Cl. C12Q 1/68

(52) U.S. Cl. 435/6

(57) ABSTRACT

Methods are provided for the determination of specific nucleotide sequences, particularly single nucleotide polymorphisms, by using probes comprising a first strand complementary to the target sequence, a shorter second strand forming a stem and a linker adjacent the double stranded stem connecting the two strands, whereby binding of target to the probe under conditions that do not cause melting of the double stranded stem in the absence of target results in the dissociation of the second strand from the first strand. The ss second strand is then detected as exemplified by using a FRET pair, where dissociation of the stem results in separation of the FRET pair and increase in fluorescence. Amplification of the target sequence may be employed prior to combination with the probe. The method finds particular application with complex nucleic acid mixtures.

### RELATED APPLICATIONS

This application is based on and claims priority of provisional application Serial No. 60/256,737, filed Dec. 19, 2000, the entire contents of which is incorporated herein by reference.

### BACKGROUND OF THE INVENTION

1. Field of the Invention

The invention relates generally to the determination of the presence of a nucleic acid sequence in a sample, particularly detecting single nucleotide polymorphisms.

2. Background Information

Determining the nucleotide sequences and expression levels of nucleic acids (DNA and RNA) is critical to understanding the function and control of genes and their relationship, for example, to disease discovery and disease management. Analysis of genetic information plays a crucial role in biological experimentation. This has become especially true with regard to studies directed at understanding the fundamental genetic and environmental factors associated with disease and the effects of potential therapeutic agents on the cell. Such a determination permits the early detection of infectious organisms such as bacteria, viruses, etc., genetic diseases such as sickle cell anemia; and various cancers. Thus, there is an increasing need within the life science industries for more sensitive and more accurate technologies for performing analysis on genetic material obtained from a variety of biological sources. The technologies should be simple, easily within the capability of a technician, substantially automatable and have a minimum number of steps involved with its performance. Unique, allelic, single nucleotide polymorphisms or mutated nucleotides or nucleotide sequences in a polynucleotide can be detected by hybridization with a nucleotide multimer, or oligonucleotide, probe. Hybridization is based on complementary base pairing.

When complementary single stranded nucleic acids are incubated together, the complementary base sequences pair to form double stranded hybrid molecules. These techniques rely upon the inherent ability of nucleic acids to form duplexes via hydrogen bonding according to Watson-Crick base-pairing rules. The ability of single stranded deoxyribonucleic acid (ssDNA) or ribonucleic acid (RNA) to form a hydrogen bonded structure with a complementary nucleic acid sequence has been employed as an analytical tool in molecular biology research. The oligonucleotide probe employed in the detection is selected with a nucleotide sequence complementary, usually exactly complementary, to the nucleotide sequence in the target nucleic acid. Following hybridization of the probe with the target nucleic acid, any oligonlucleotide probe/nucleic acid hybrids that have formed are detected by a change in a signal associated with a label attached to the probe or by separation from unhybridized probe whereupon the amount of oligonucleotide probe bound to the target is then tested to provide a qualitative or quantitative measurement of the amount of target nucleic acid originally present.

One method for detecting specific nucleic acid sequences generally involves immobilization of a target nucleic acid on a solid support such as nitrocellulose paper, cellulose paper, diazotized paper, a nylon membrane, beads, plastic surfaces and so forth. After the target nucleic acid is fixed on the support, the support is contacted with a suitably labeled nucleic acid for about two to forty-eight hours. After the above time period, the solid support is washed several times at a controlled temperature to remove unhybridized probe. The support is then dried and the hybridized material is detected by autoradiography or by spectrometric methods. Such approaches are often referred to as heterogeneous assays because they involve separation of free and bound material such as separation of probes that are hybridized to a target polynucleotide and unhybridized probes.

Another method for detecting specific nucleic acid sequences employs hybridization to surface-bound arrays of sample nucleic acid sequences or oligonucleotide probes. Such techniques are useful for analyzing the nucleotide sequence of target nucleic acids. Hybridization to surface-bound arrays can provide a relatively large amount of information in a single experiment. For example, array technology has identified single nucleotide polymorphisms within relatively long (1,000 residues or nucleotides) sequences. In addition, array technology is useful for some types of gene expression analysis, relying upon a comparative analysis of complex mixtures of mRNA target sequences.

Homogeneous assays are also known for analyzing nucleic acids. The assays are referred to as homogeneous in that they do not normally involve separation of bound and free material. Such assays utilize various labels such as fluorescent labels and label systems such as fluorescent label pairs or fluorescers in conjunction with quenchers. Many of these known assays use at least two probes for each target polynucleotide.

As mentioned above, detection of a target polynucleotide sequence usually entails binding one or more oligonucleotide probes to the target polynucleotide. By selection of an appropriate level of stringency, the oligonucleotide probe will bind only a specific sequence. However, the stringency often must be tailored for a given probe-target pair, particularly when the target must be distinguished from a like sequence differing by only one nucleotide at a polymorphic site.

Numerous methods are known that attempt to address the above problem and to achieve adequate selectivity. In one approach, arrays of probes are used in which four probes are used for a given target polynucleotide sequence. The four probes differ only by having each of the four nucleotides present at the polymorphic site.

In another approach, the oligonucleotide probe can be a primer that binds adjacent to the polymorphism and is only extended in the presence of the appropriate nucleotide triphosphate complementary to the polymorphic nucleotide. Alternatively, two oligonucleotide probes have been employed that can bind at adjacent sites on the target and abut each other at the polymorphic site. Upon treatment with a ligase the probes become ligated to each other only if they exactly match the target. Still another method employs a 5′-nuclease that cleaves an oligonucleotide probe that has an unhybridized 5′-end when bound to the target adjacent the 3′-end of a second bound oligonucleotide but fails to cut when there is a base mismatch between the probe and the target adjacent the second bound oligonucleotide.

In all of these methods it is necessary to use multiple probes and/or nucleic acid amplification primers, and the stringency of the reaction conditions must be very precisely controlled to achieve the desired detection specificity. When high levels of multiplexing are desired the use of multiple oligonucleotide probes and primers becomes very costly, and it becomes particularly difficult to identify multiple oligonucleotide probe sequences that will all hybridize selectively to a set of target polynucleotides under a standard set of conditions. There is therefore a need for a method that will permit highly specific detection of nucleotide sequences with a minimum number of oligonucleotide probes where tight control of the assay conditions is not a prerequisite. Additionally, it is desirable that such probes can be readily designed to provide sufficient specificity for detection of single base differences in a target polynucleotide without the need for complex algorithms.

Other prior art techniques employing hairpin probes may be found in U.S. Pat. Nos. 4,725,537; 4,766,062; 4,795,701; 5,770,365; 5,866,336; 5,925,517; 6,025,133 and 6,037,130, hereby incorporated by reference.

All patents, patent applications or published references cited herein are hereby incorporated by reference.

### SUMMARY OF THE INVENTION

The present invention relates to the accurate detection of at least one nucleic acid sequence in a sample, particularly the presence of a single nucleotide polymorphism, using the method comprising incubating the medium with a probe comprising (1) a first oligonucleotide sequence that is complementary to the target polynucleotide (the long strand), (2) a second oligonucleotide sequence that is complementary to and hybridized with a portion of the first oligonucleotide sequence (the short strand) thereby creating a hybridized region and a single stranded region of the first oligonucleotide sequence, and (3) a linker connecting said first and second oligonucleotide sequences. The hybridization of the hybridized region of the first oligonucleotide sequence with the target polynucleotide takes place under conditions that do not cause spontaneous dissociation of the double stranded stem in the absence of target and proceeds with strand displacement of the short strand. The displaced short strand is detected and is related to the presence of the target polynucleotide. The target nucleic acids may have been subject to amplification prior to detection. The dissociation of the hybridized region is detected by a variety of techniques. The probe may be in solution or bound to a surface.

### BRIEF DESCRIPTION OF THE DRAWINGS

FIG. 1 is a schematic diagram depicting one embodiment of a probe in accordance with the present invention.

FIG. 2 is a schematic diagram depicting another embodiment of a probe in accordance with the present invention.

FIG. 3 is a schematic diagram depicting another embodiment of a probe in accordance with the present invention.

FIG. 4 is a schematic diagram depicting another embodiment of a probe in accordance with the present invention.

FIG. 5 is a schematic diagram depicting another embodiment of a probe in accordance with the present invention.

FIG. 6 is a schematic diagram depicting an embodiment of a method in accordance with the present invention.

FIG. 7 is a schematic diagram depicting another embodiment of a method in accordance with the present invention.

FIG. 8 is a schematic diagram depicting another embodiment of a method in accordance with the present invention.

FIG. 9 is a graph of probe melting curves having mismatches at different positions.

FIGS. 10A and B are graphs of fluorescence increase as a result of changes in concentration of target nucleic acid and mismatched target, respectively, to a probe comprising a FRET pair.

FIGS. 11A and B are graphs of increases in fluorescence determined kinetically and concentration related, respectively.

FIG. 12 is a graph of fluorescence response to the presence of matched and mismatched targets.

### DETAILED DESCRIPTION OF THE INVENTION

Methods and compositions are provided for identifying at least one nucleic acid sequence in a complex nucleic acid sample employing a probe, that may be referred to as a stem and loop (stem-loop) or hairpin that is characterized by having 1) a first oligonucleotide sequence that is complementary to the target polynucleotide (the long strand), (2) a second oligonucleotide sequence that is complementary to and hybridized with a portion of the first oligonucleotide sequence (the short strand) thereby creating a hybridized region and a single stranded region of the first oligonucleotide sequence, and (3) a linker connecting the first and second oligonucleotide sequences. Binding of the target sequence to a complementary probe results in strand displacement of the short strand. The hybridizing conditions are selected so that dissociation of the short strand from the long strand occurs almost solely from strand displacement by target. Displacement of the short strand is detected as indicative of the presence of the target sequence present in the sample. A single nucleotide difference between the target sequence and the short strand can be readily detected due to the substantial absence of displacement by the target nucleic acid of the short strand and the absence of dissociation when target is not bound.

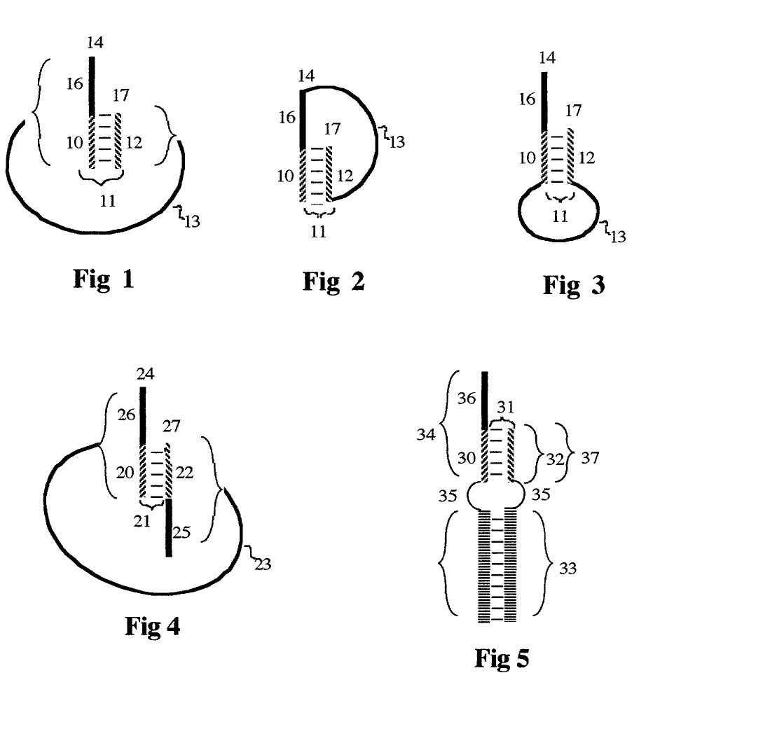

The probes that are used in the present methods (referring to FIGS. 1-3) comprise (1) a first oligonucleotide sequence 14 (the long strand) that is complementary to a target polynucleotide and is comprised of a hybridized region 10 and a single stranded region 16 (2) a second polynucleotide sequence 17 (the short strand) that is comprised of a complementary region 12 which is complementary with and can hybridize to the hybridized region 10 of the first oligonucleotide sequence and (3) a linker 13 that provides irreversible binding or attachment of the first and second oligonucleotide sequences under the conditions of the method of this invention. Accordingly, the probes are comprised of a hybridized portion 11 consisting of the duplex produced by hybridization of hybridized region 10 of the first oligonucleotide sequence and complementary region 12 of the second oligonucleotide sequence (the stem). The hybridized portion is depicted by the cross-hatched lines between the oligonucleotide sequences.

The oligonucleotide sequences that comprise a portion or all of the probes of the invention may be natural polynucleotides, that is, comprised of natural nucleotides such as ribonucleotides and deoxyribonucleotides and their derivatives that contain the usual four nucleotides or bases, namely, T (or U), G, A and C. Alternatively, the oligonucleotide sequences may be unnatural polynucleotides comprised of nucleotide mimetics such as, for example, protein nucleic acids (PNA), 2′O-modified nucleosides and oligomeric nucleoside phosphonates. The oligonucleotide sequences may also be a combination of natural and unnatural nucleotides.

The design of the probes depends on where the probe binds to the target polynucleotide relative to suspected sequence differences in the samples. In general, the length of the hybridized portion and the single stranded region of the probes of the invention depend on the hybridization conditions that are to be used. For example, long single stranded regions are required to permit hybridization when higher temperatures are needed to avoid interference due to formation of secondary structures of a target polynucleotide. When it is desired to avoid spontaneous dissociation of the strands at higher temperatures, longer double stranded portions of the probe will be required. The first oligonucleotide sequence generally has a hybridized region of about 5 or more, usually about 8 or more nucleotides, and usually less than about 35, more usually, less than about 20 nucleotides, that are complementary to the second sequence. While longer sequences may be employed they are disadvantageous in requiring the synthesis of larger molecules. There is no critical upper limit to the number of nucleotides in the hybridized region other than any practical problems associated with preparing very long probes. The length of the single stranded region of the first oligonucleotide sequence is usually at least about 6 nucleotides and may be at least about 15 or more nucleotides, generally not being more than about 30. The subject invention provides high specificity for the polynucleotide sequence with a relatively small probe, conveniently 35 nucleotides or fewer, generally not fewer than 17 nucleotides, excluding any nucleotides in the linker or loop. Practical considerations will generally have the single stranded tail portion of the hairpin in the range of about 11 to 23 nt, the stem will generally be in the range of about 6-20 nt, and the loop, when it is an oligonucleotide will generally be in the range of about 3 to 30 nt.

Short single stranded regions, preferably fewer than about 20 nucleotides, will be preferred when mismatches are suspected in the portion of the target complementary to the single stranded region. When mismatches are suspected in the portion of the target polynucleotide complementary with the hybridized region, there is no critical upper limit to the number of nucleotides in either the single stranded region or double stranded stem other than matters of practicality.

The complementary sequence of the second oligonucleotide sequence is identical in length with the hybridized region of the first oligonucleotide sequence. The second oligonucleotide sequence may also comprise a single stranded portion contiguous with the complementary sequence and at the opposite end of the hybridized portion of the probe from the single stranded region of the first oligonucleotide sequence. Thus, the 3′-ends of both the first and second oligonucleotides or the 5′-ends of both of the oligonucleotides form the hybridized portion of the probe with the single stranded portions of each of the sequences extending from opposite ends of the hybridized portions. That is, the long strand may have an unbonded terminus that is 5′O or 3′O, with the unbonded terminus of the short strand being the reciprocal, namely 3′O or 5′O respectively. Preferably, the long strand has a 3′O-terminus proximal to the linker.

The composition of these probes is better understood by reference to FIG. 4, which illustrates (1) a first oligonucleotide sequence 24 that is complementary to a target polynucleotide, having the opposite orientation from the first oligonucleotide sequence, and is comprised of a hybridized region 20 and a single stranded region 26, (2) a second polynucleotide sequence 27 that is comprised of a complementary region 22, which is complementary with and can hybridize to hybridized region 20 of the first oligonucleotide sequence 24 and a single stranded portion 25, and (3) a linker 23 that provides irreversible attachment of the first and second oligonucleotide sequences under the conditions of the method of this invention. Accordingly, these probes are comprised of a hybridized portion 21 consisting of the duplex produced by hybridization of the first and second oligonucleotide sequences 24 and 27.

Although not necessary in conducting the present methods, when a method of this invention is carried out in the presence of a polymerase and nucleotide triphosphates, the 3′-ends of the two-oligonucleotide sequences of the probe are preferably blocked to prevent polymerase catalyzed extension. Blocking can be achieved in any convenient manner that prevents chain extension. Such approaches include, for example, attachment to the 3′-end of a phosphate, a ribonucleotide, a dideoxynucleotide, an abasic ribophosphate, an unnatural base, a polymer, a chemical linkage to a surface or to the linker, or one or more natural bases that do not hybridize to the other strand of the probe, the target polynucleotide, or any reference polynucleotide. Such an end group can be introduced at the 3′ end during solid phase synthesis or a group can be introduced that can subsequently be modified. For example, in order to introduce dextran at the 3′-end, a ribonucleotide can be introduced at the 3′-end and then oxidized with periodate followed by reductive amination of the resulting dialdehyde with borohydride and aminodextran. The details for carrying out the above modifications are well known in the art and will not be repeated here.

When a method of this invention is carried out in the presence of a 5′-nuclease it may be necessary to protect one or more bases near the 5′-ends of the oligonucleotide sequences from degradation. This can conveniently be achieved, for example, by incorporating phosphorothioates, phosphonates, or other enzymatically inert groups in place of the phosphate diesters of the oligonucleotides. Alternatively, attachment of the linker to each of the 5′-ends will prevent degradation by certain 5′-nucleases. The linker is a group involved in the irreversible attachment or binding or linkage of the first and second oligonucleotide sequences. The linkage may be covalent or non-covalent. When the linkage is non-covalent the linker will usually comprise a duplex of two complementary nucleic acid strands, each covalently attached to one of the oligonucleotide sequences. The duplex comprises sequences that do not dissociate during the use of the probe in the present method. This may be accomplished by constructing a duplex that is long enough to avoid melting under the intended assay conditions. Preferably, the duplex has a relatively high G/C content or is double stranded RNA or is comprised of PNA.

When the linker is covalent, it may be a bond but is usually a group that is polymeric or monomeric and comprises a bifunctional group convenient for linking the two sequences. The linkers may be hydrophilic or hydrophobic, preferably hydrophilic, charged or uncharged, preferably uncharged, and may be comprised of carbon atoms and heteroatoms, such as oxygen, nitrogen, phosphorous, sulfur, etc. In this invention, the linker need not be an oligonucleotide, although oligonucleotides may be used, where the sequence may be designed for sequestering the probe, binding of a labeled complementary sequence, or other means of identification. Alternatively, the sequence when other than an oligonucleotide, may be aliphatic, alicyclic, aromatic, heterocyclic, or combinations thereof, particularly aliphatic, being a chain of from about 5 to 25 atoms, allowing flexibility in the probe, and keeping the two polynucleotide strands together.

The linkers may be monomeric or polymeric, where the termini will have functionalities that allow for binding, usually bonding, to the long and short strands. Polymeric linkers may comprise, for example, an oligonucleotide or related polyalkenylphosphate, a polypeptide, a polyalkylene glycol, e.g. polyethylene glycol, and the like. Monomeric linkers may comprise, for example, alkylenes, ethers, amides, thioethers, esters, ketones, amines, phosphonates, sulfonamides, and the like. Where the linker comprises an oligonucleotide sequence that is part of the chain of atoms linking the first and second oligonucleotide sequences, at least a portion of such sequence will usually not become hybridized to a target polynucleotide when carrying out methods of this invention.

The two ends of the linker are attached covalently to the first and second oligonucleotide sequences, respectively, in a manner that does not interfere with hybridization capabilities of the two sequences. Thus, the linker may be linked to any nucleotide or a terminus of each oligonucleotide sequence (see FIG. 1). When attachment is to a non-terminal nucleotide, it frequently is at the 5-position of U or T, the 8-position of G, the 6-amino group of A, a phosphorus atom, or the 2′-position of a ribose ring. Usually, it is most convenient to attach the linker to one of the termini of each oligonucleotide sequence. Attachment to the same terminus of each sequence will often be convenient when the linker is not an oligonucleotide (see FIG. 2). Attachment at the opposite termini, that is, the 3′-end of one oligonucleotide sequence and the 5′-end of the other oligonucleotide sequence, is convenient when the linker is an oligonucleotide or polyalkenylphosphate (see FIG. 3).

One embodiment of a probe illustrated in FIG. 3 is shown in more detail in FIG. 5. As discussed above, the linker may be non-covalent and comprised of a nucleic acid duplex provided that it is formulated so that it does not dissociate when carrying out the methods of this invention. FIG. 5 shows (1) a first oligonucleotide sequence 34 that is complementary to a target polynucleotide and is comprised of a hybridized region 30 and a single stranded region 36, (2) a second polynucleotide sequence 37 that is comprised of a complementary region 32 which is complementary with and can hybridize to the hybridized region 30 of the first oligonucleotide sequence and (3) a linker 33 that comprises a nucleic acid duplex and bivalent connectors that are not self-complementary nucleotides 35, that provides irreversible attachment of the first and second oligonucleotide sequences under the conditions of the method of this invention. The sequence of the nucleic acid duplex is unrelated to sequences comprising the target polynucleotide. The probe is, thus, comprised of a hybridized portion 31 that is contiguous with the nucleic acid duplex comprising the linker 33 and that consists of hybridized region 30 of first oligonucleotide sequence 34 and complementary region 32 which, in the embodiment depicted in FIG. 5, is identical to second oligonucleotide sequence 37. By providing short bivalent connectors, 35, comprised of chais of one to five atoms it is possible to maintain close proximity of the first and second oligonucleotide sequences 34 and 37 after hybridization of the first oligonucleotide sequence 34 with a target polynucleotide. Maintaining proximity is desirable where there may be a single base mismatch in the hybridized region 30 and the target polynucleotide. By maintaining close proximity of sequence 34 and 37, any inaccurate hybridization is more likely to be reversed than if sequences 34 and 37 become spatially separated.

Common functionalities that may be used in forming a covalent bond between the linker and the nucleotide of the sequences to be conjugated are alkylamine, amidine, thioamide, ether, urea, thiourea, guanidine, azo, thioether and carboxylate, sulfonate, and phosphate esters, amides and thioesters. Various methods for linking molecules are well known in the art; see, for example, Cuatrecasas, J. Biol. Chem. (1970) 245:3059. Probes of this invention comprise a label. The function of the label is to permit detection of dissociation of the hybridized portion of the probe upon binding to a target polynucleotide. The label may be an intrinsic part of one or both of the oligonucleotide sequences of the probe or the linker or may be attached to the probe. For some modes of detection it may be necessary to incorporate two or more labels in the probe.

In carrying out the method for detecting the target oligonucleotide, the event that is measured is the disassociation of the second oligonucleotide from the first oligonucleotide, so that the second oligonucleotide is now single stranded and distal from the first oligonucleotide. The disassociation resulting from strand displacement may be measured in many ways, particularly, using a variety of labels or detection techniques relevant to the disassociation and presence of the single stranded second oligonucleotide.

Detection can be by a homogeneous method that provides for a change in the nature of a signal from a label or by a heterogeneous method that is based on separation of a bound from an unbound substance in the medium. Labels include ligands and their complementary receptors; surfaces including solid supports and dispersible beads; detectable labels; and chemically reactive groups that can be converted or linked to ligands, surfaces or detectable labels. Detectable labels include any group that permits detection of a binding or dissociation event such as isotopic and non-isotopic labels; dyes; fluorescent, chemiluminescent, and electroluminescent labels, particularly ruthenium chelates; quenchers capable of changing the emission properties of a luminescent label; mass tags for changing the molecular weight for detection by mass spectroscopy or acoustic wave perturbation; particles such as latex beads, dye crystallites, carbon particles, liposomes, metal sols, and the like; electroactive groups; magnetic materials, particularly super paramagnetic and ferromagnetic particles; spin labels; catalysts such as enzymes, coenzymes, and photosensitizers; enzyme inhibitors and activators including enzyme fragments capable of complementation to form holoenzymes, particularly enzyme fragments, e.g. enzyme donors, derived from α-galactosidase and ribonuclease, transcription factors, and the like.

Fluorescent labels are particularly useful and are well known in the art. Typical labels include coumarins such as umbelliferone, bimanes, xanthenes such as fluorescein, rhodamine, and their derivatives, cyanines, oxazines, phthalocyanines, phycobiliproteins, squaraines, and the like. Quenchers which are frequently used in combination with a fluorescer for fluorescence resonance energy transfer detection (FRET) are likewise well known in the art and include any of the aforementioned fluorescent labels, non-fluorescent dyes such as DABCYL, hydroxyfluoresceins, azo-compounds, electron donors such as anilines and other amines, electron acceptors such as quinones, and the like. Chemiluminescent labels include cyclic and acyclic acylhydrazides such as luminol, natural and synthetic luciferins, acridium esters, dioxetanes, oxalate esters, etc.

In a broader context, one may think of molecular energy transfer (MET) as described in U.S. Pat. No. 6,090,552, whose disclosure beginning at column 16, line 48 and continuing to column, 17, line 10, and beginning at column 18, line 48 and continuing to column 20, line 20, is specifically incorporated by reference as if it were set forth herein. With MET, as in the case of the special case of FRET, disassociation of the second oligonucleotide from the first oligonucleotide results in inhibition of energy transfer, so that the observed signal is different in the case of the associated first and second oligonucleotides as compared to the disassociated state. For example, in the case of FRET, one may observe the absence of fluorescence when the two strands are associated or a change in the emission frequency when the two strands are associated, depending upon the selection of the members of the FRET pair.

Ligands include such ligands as biotin and folate that have natural receptors and haptens that have complementary antibodies. Groups capable of being converted or bound to ligands, detectable labels or surfaces may be any chemically active group distinguished from other groups in the probe such as photoactivatable groups, glycols, amines, aldehydes, acids, esters, electrophilic groups such as a-haloketones, a-haloamides, monomers capable of polymerization, and the like, wherein the group is capable of coupling with a specific group of a ligand or detectable label. Thus, for example, glycols can be converted to di-aldehydes with periodate and aldehydes can be coupled with amines on a label by reductive alkylation. Similarly, electrophilic groups can be coupled with amines, sulfhydryl groups, and phenols, etc., that are attached to a label; amino acids can be converted to fluorescent groups with fluorescamine; acids, active esters, and other electrophiles can be coupled to amines on surfaces or other labels, etc.

A simple form of the label is the dissociated second or short strand oligonucleotide sequence itself. Upon binding of the target polynucleotide to the probe and strand displacement, this sequence is no longer hybridized and becomes single stranded. There are various methods for detecting the formation of this single stranded sequence. If the target and the single stranded region of the first oligonucleotide are RNA and the second oligonucleotide is DNA, a single stranded DNA hydrolase such as S1 nuclease degrades the second oligonucleotide of the probe only after strand displacement. The degradation products can be detected by coupling to appropriate enzymes that act on nucleotide monophosphates, by mass spectroscopy, HPLC, and the like. Similarly, the single stranded region of the probe and the target polynucleotide can be DNA and the second oligonucleotide can be RNA. An enzyme such as ribonuclease A that hydrolyses single stranded RNA can then be used together with one of the aforementioned methods of detecting nucleotide monophosphates to detect strand displacement. Still another approach is to detect strand displacement by use of a support, which has a sequence complementary to the second oligonucleotide. Only probe:target complexes that have undergone strand displacement bind to the support. Binding of the complex to the support can then be detected provided the probe or target polynucleotide is labeled with a detectable label such as, for example, a luminescent group, an enzyme, metal particle, latex bead, or a radioactive group. Labeling of the target polynucleotide can be accomplished by well-known methods such as PCR, labeling with a second probe, nick translation, etc. Upon binding of the complex to the support, the amount of label attached to the support is determined, usually following separation of the support from unbound components.

The hybridized region of the first oligonucleotide sequence may also serve as a label. For example, the hybridized region of the probe can comprise double stranded RNA when the target polynucleotide is DNA. Upon binding of the target to the probe and strand displacement, a DNA:RNA heteroduplex is formed. In the presence of ribonuclease H, the RNA in the heteroduplex is hydrolyzed and the hydrolysis fragments can be detected. This method is similar to the detection method previously described for conventional probes by Duck, U.S. Pat. No. 5,011,769, the relevant disclosure of which is incorporated herein by reference.

The linker can also serve as a label. For example, the linker can be an oligonucleotide sequence that is incapable of binding to a complementary sequence when both ends are attached to the hybridized portion of the probe. However, upon dissociation of the second oligonucleotide from the hybridized region of the first oligonucleotide, the linker is no longer part of a ring and can then hybridize to a complementary sequence attached to a support. As already described above, binding of the complex to a support can be detected provided that the probe or the target polynucleotide has a detectable label. Alternatively, the linker can be a chain of atoms that produces a change in a signal upon strand displacement and ring opening. One such sequence is a polypeptide that can complement with an incomplete enzyme to form a holo-enzyme more efficiently when in the ring opened form. One example of such a polypeptide is a 45-90 amino acid fragment of â-galactosidase. The two oligonucleotide sequences of the probe can be bound to this linker through sulfhydryl groups that are introduced into the polypeptide as cysteines. The resulting probe complements relatively inefficiently with the remaining portion of the â-galactosidase molecule known as an enzyme acceptor. Upon dissociation of the hybridized portion of the probe, complementation is facilitated and detected as an increase in enzyme activity. Complementation of â-galactosidase fragments and their use in detection of binding events is further described by Henderson, U.S. Pat. No. 4,708,929, the relevant disclosure of which is incorporated herein by reference.

Frequently, probes of this invention comprise labels that are covalently attached to the oligonucleotide sequences or the linker. Detection of the probe:target polynucleotide complexes produced in this fashion is similar to detection of common linear probe:target polynucleotide complexes. The probe may be labeled with a ligand such as biotin that facilitates its binding to a support. When the probe is complexed with a detectably labeled target polynucleotide, the detectable label becomes affixed to the support and can conveniently be detected following separation and washing of the support. Alternatively, the probe can be labeled with a detectable label and the target polynucleotide can be labeled with a ligand. The labels may be those described above. To summarize, a large variety of detectable labels are well known including labels that are detectable by electromagnetic radiation, electrochemical detection, mass spectroscopic measurements, acoustic wave detection and the like. Among these fluorescent and chemiluminescent labels are frequently preferred for detection of nucleic acid binding, but other modes of detection can also be used with the probes of this invention.

In another application of labels that are covalently attached to the probes, the labels are designed to produce a signal that is modulated as a result of strand displacement. Various strategies have previously been described for detecting hybridization or dissociation of two nucleic acid strands. For example, acridinium esters can be attached to a base in the second oligonucleotide of the probe in a manner that causes the acridinium group to be protected from reaction with peroxide only so long as the second oligonucleotide remains hybridized. Upon strand displacement the acridinium ester is no longer protected, reaction occurs with the peroxide and light is emitted. This type of label system is described by Arnold, U.S. Pat. No. 5,283,174, the relevant disclosure of which is incorporated herein by reference. Similarly, a fluorescent label can be attached to an oligonucleotide in a manner that causes it to intercalate into a double strand formed upon hybridization to a complementary sequence. Intercalation typically causes an increase in fluorescence, which can be used to monitor the extent of hybridization.

Another method for detection of dissociation of the hybridized portion is by the use of a luminescent label on one strand of the hybridized portion and a label that causes quenching of the luminescence on the other strand. Upon dissociation of the hybridized portion of the probe the labels become separated and the emission increases. Fluorescence quenching caused by proximity of an energy acceptor or other type of quencher has been extensively studied and used in many analytical applications. Examples of this method, including means of attachment and appropriate sites of attachment to probes, are described more fully U.S. Pat. Nos. 4,996,143, 5,565,322 (column 9, line 37, to column 14, line 7) and U.S. Pat. No. 6,037,130, the relevant disclosure of which are incorporated herein by reference.

Still another method for detection of strand displacement is by changes in the polarization of fluorescence of a fluorescent label attached to the second oligonucleotide. Upon dissociation of the hybridized portion of the probe, the second oligonucleotide becomes single stranded and, therefore, can more freely rotate leading to depolarization of its fluorescence emission. Fluorescent labels that have relatively long lived excited states are preferred for this mode of detection such as, for example, ruthenium and lanthanide chelates and pyrene.

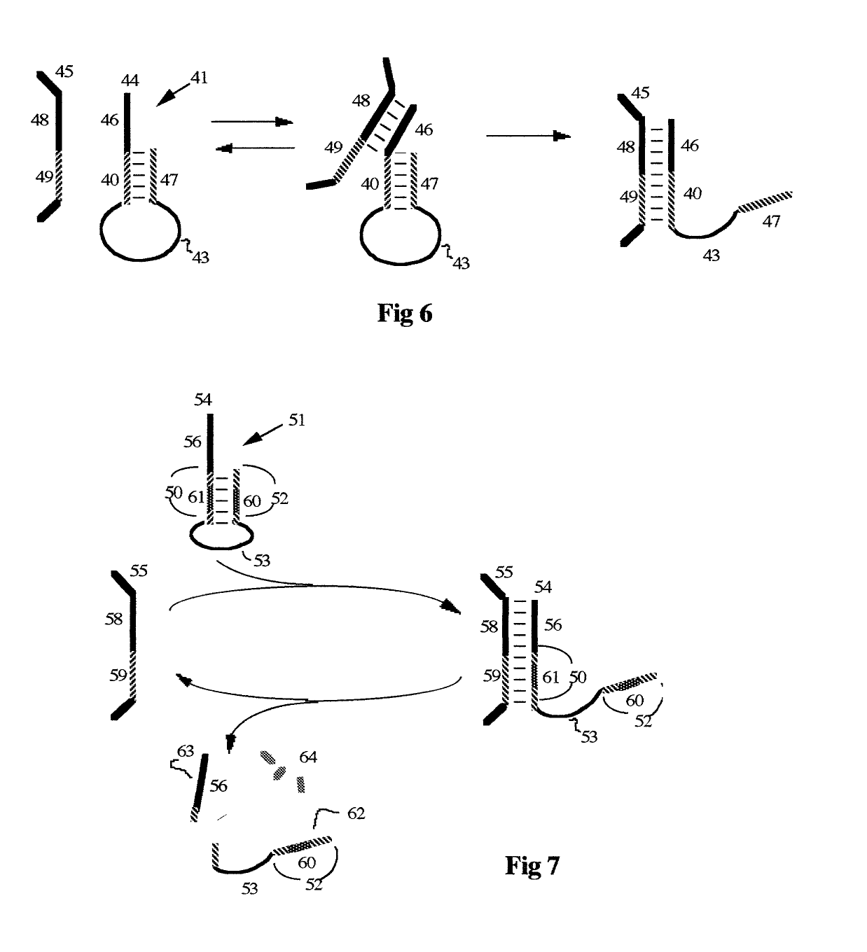

As mentioned above, the aforementioned probes may be employed in methods for determining a target polynucleotide. Referring to FIG. 6, one method comprises hybridizing a target polynucleotide 45 with a probe 41 of the invention under conditions wherein the second oligonucleotide sequence 47 of the probe remains hybridized to the hybridized region 40 of the first oligonucleotide sequence 44 in the absence of the target polynucleotide. Probe 41 also comprises a single stranded region 46 of the first oligonucleotide sequence 44, which is complementary to portion 48 of target polynucleotide 45, and a linker 43, which links the first and second oligonucleotide sequences. Upon incubation of probe 41 with the target polynucleotide, the single stranded region 46 hybridizes with the target at portion 48 to form a duplex indicated by cross-hatching. Provided an incubation temperature is used that is near the melting point of this duplex, probe 41 remains substantially bound to the target polynucleotide only if portion 49 of the target polynucleotide is complementary to the hybridized region 40 of the first oligonucleotide. If sequences 40 and 49 are not complementary the probe dissociates from the target polynucleotide. If they are complementary, strand displacement takes place leading to complete hybridization of target polynucleotide with regions 40 and 46 of the first oligonucleotide sequence and release from hybridization of the second oligonucleotide sequence 47. Complementarity of target polynucleotide portion 49 and hybridized region 40 may then be detected by determining either the binding of probe 41 to the target polynucleotide or by the dissociation of the second oligonucleotide sequence 47 from the hybridized region 40.

The target polynucleotide is a polymeric nucleotide or nucleic acid polymer and may be a natural compound or a synthetic compound. The target polynucleotide can have at least about 15 more usually at least about 30 nucleotides and may comprise any higher number of nucleotides. The target polynucleotides include nucleic acids, and fragments thereof, from any source in purified or unpurified form including DNA (dsDNA and ssDNA) and RNA, including tRNA, mRNA, rRNA, mitochondrial DNA and RNA, chloroplast DNA and RNA, DNA/RNA hybrids, or mixtures thereof, genes, chromosomes, plasmids, cosmids, the genomes of biological material such as microorganisms, e.g., bacteria, yeasts, phage, chromosomes, viruses, viroids, molds, fungi, plants, animals, humans, and the like. The target polynucleotide can be only a minor fraction of a complex mixture such as a biological sample. Also included are genes, such as hemoglobin gene for sickle-cell anemia, cystic fibrosis gene, oncogenes, cDNA, and the like.

The target polynucleotide can be obtained from various biological materials by procedures well known in the art. A polynucleotide, where appropriate, may be cleaved to obtain a fragment that is the target polynucleotide, for example, by shearing or by treatment with a restriction endonuclease or other site-specific chemical cleavage method. The target polynucleotide may be generated by in vitro replication and/or amplification methods such as the Polymerase Chain Reaction (PCR), asymmetric PCR, the Ligase Chain Reaction (LCR), transcriptional amplification by an RNA polymerase, rolling circle amplification, strand displacement amplification (SDA), NASBA, and so forth. The target polynucleotides may be either single-stranded or double-stranded. A target polynucleotide may be treated to render it denatured or single stranded by treatments that are well known in the art and include, for instance, heat or alkali treatment, or enzymatic digestion of one strand. In the present invention the identity of the target polynucleotide should be known to an extent sufficient to allow preparation of a sequence hybridizable with the target polynucleotide. Normally the target polynucleotide will be present in low concentrations in the sample, usually less than micromolar and frequently less than picomolar. The lower limit of detection of the methods of this invention will dictate the lowest concentrations of target polynucleotide that can be used.

Normally, the target polynucleotide to be analyzed must be extracted from a biological sample.. Such samples include biological fluids such as blood, serum, plasma, sputum, lymphatic fluid, semen, vaginal mucus, feces, urine, spinal fluid, and the like; biological tissue such as tissue biopsies, hair and skin; and so forth. Other samples include cultures of mammalian and non-mammalian cells, microorganisms, viruses, yeast, fungi, and the like, plants, insects, aquatic organisms, food, forensic samples such as paper, fabrics and scrapings, water, sewage, medicinals, etc. When necessary, the sample may be pretreated with reagents to liquefy the sample and release the nucleic acids from binding substances. Such pretreatments are well known in the art. Hybridization of a probe with a target polynucleotide will usually be carried out at temperatures below the temperature at which the hybridized region of the probe spontaneously dissociates, usually, at about 5 to about 80° C., more usually, at about 20 to 70° C. The higher temperatures within the above ranges may be used particularly when the probes of the invention are used for monitoring amplification reactions involving thermal cycling. The hybridizing is carried out under conditions wherein the second oligonucleotide sequence remains hybridized to the first oligonucleotide sequence in the absence of the target polynucleotide in order to obtain the highest binding specificity. The probe and target polynucleotide combination generally is incubated under conditions suitable for hybridization of the first oligonucleotide sequence with the target polynucleotide, below the melting temperature of the hybridized portion of the probe, and under conditions where strand displacement will occur upon the binding of the single stranded region of the probe to the target polynucleotide followed by displacement of the second oligonucleotide sequence by the target polynucleotide.

Incubation times can vary from less than a minute to several hours or more depending on the concentration of the reactants, the temperature, the type of buffer, etc. The concentration of the probes required for hybridization with target polynucleotides in the present method may be relatively high to provide rapid binding, generally as high as about 100 micromolar, but usually no higher than about 10 micromolar and frequently as low as 1 micromolar. Where assay speed is unimportant, much lower concentrations of the probes may be used, usually as low as 1 pM, but generally no lower than 100 pM. The probes will usually be at least equal to the estimated concentration of the target sequence and generally in at least about 2-fold excess, frequently at least about 5-fold excess or greater.

In carrying out the present method, an aqueous medium is employed. Other polar cosolvents may also be employed, usually oxygenated organic solvents of from 1-6, more usually from 1-4, carbon atoms, including dimethylsulfoxide, alcohols, ethers, formamide and the like. Usually these cosolvents, if used, are present in less than about 70 weight percent, more usually in less than about 30 weight percent.

The pH for the medium is usually in the range of about 4.5 to 9.5, more usually in the range of about 5.5-8.5, and preferably in the range of about 6-8. Various buffers may be used to achieve the desired pH and maintain the pH during the determination. Illustrative buffers include borate, phosphate, carbonate, Tris, barbital and the like. A metal ion such as magnesium ion is usually present in the above medium.

It should be noted that the methods in accordance with the present method do not require a nucleotide polymerase. Accordingly, the present methods may be conducted in the absence of a polymerase. However, there are some circumstances where a nucleotide polymerase might be present in the reaction medium, such as where the present probes are employed to monitor an amplification reaction such as PCR. Normally, the nucleotide polymerase is necessary only for the amplification reaction and does not participate in the performance of the present probes. Frequently, extension or degradation of the present probes may impair their performance and it will be necessary to prevent the probes from being extended by the polymerase or degraded by associated nuclease activity as described above.

Following or concurrent with the incubation of the probe and the target polynucleotide, the dissociation of the second oligonucleotide sequence from the hybridized region of the probe is detected and is related to the presence or amount of the target polynucleotide in the sample. Detection may be achieved by employing a label or label system as discussed above. Measurement of the signal generated as a result of the present method is accomplished by an approach commensurate with the type of label or label system. Such measurement approaches are well known in the art and will not be repeated here.

In one embodiment of the present invention, the probes of the present invention provide a method for amplification of the signal produced in response to binding of the probe to a target polynucleotide. The process usually is based on the use of a probe with a hybridized portion comprised of double stranded RNA. In this embodiment binding of the hybridized region of the first oligonucleotide sequence to a target polydeoxynucleotide leads to formation of a heteroduplex comprising the target polydeoxynucleotide and at least a portion of the hybridized region of the first oligonucleotide sequence. RNAse H, which is included in the reaction mixture, catalyses enzymatic degradation of the hybridized region resulting in release of the single stranded region of the first oligonucleotide sequence, the second oligonucleotide sequence and fragments of the hybridized region. The probe is employed in excess concentration over the suspected concentration of the target polydeoxynucleotide. Subsequent hybridization of another molecule of probe with a target polydeoxynucleotide molecule followed by degradation of the hybridized region results in the production of multiple molecules of the degradation products. The process continues under isothermal conditions giving a linear amplification of degradation product. One or more of these degradation products is detected and related to the presence of the target polynucleotide in the medium. Detection may be accomplished by utilizing a label or label system as discussed above. Usually, the probes used in this procedure will have a hybridized portion comprised of double stranded RNA, the long strand of which is complementary to a DNA target polynucleotide sequence.

Referring to FIG. 7, target polydeoxynucleotide 55 is combined with probe 51. Probe 51 comprises a second oligonucleotide sequence 52, which is complementary to a hybridized region 50 of a first oligonucleotide sequence 54. An RNA sequence 60 comprises a portion of the second oligonucleotide sequence 52 and is complementary with an RNA sequence 61 of hybridized region 50. The first oligonucleotide sequence 54 also has a single stranded region 56. Hybridized region 50 is complementary to portion 59 of target polydeoxynucleotide 55, and single stranded region 56 of the first oligonucleotide sequence 54 is complementary to portion 58 of target polydeoxynucleotide 55. Probe 51 also comprises linker 53, which links the first and second oligonucleotide sequences.

The method comprises hybridization of the target polynucleotide 55 with probe 51 under conditions where the second oligonucleotide sequence 52 remains hybridized to the first oligonucleotide sequence 54 in the absence of target polydeoxynucleotide 55. In the presence of target polydeoxynucleotide 55, probe 51 hybridizes with the target polydeoxynucleotide, and a heteroduplex is formed between the target polydeoxynucleotide and the RNA sequence 61 of the probe 51 with concomitant dissociation of the second oligonucleotide 52 from the hybridized region 50 of the first oligonucleotide 54. In the presence of RNAse H, which can hydrolyze DNA:RNA heteroduplexes, the RNA sequence 61 is then cleaved. The resulting fragments of the first oligonucleotide sequence 54 are too short to remain bound to target polynucleotide 55 or to the second oligonucleotide sequence 52 and the complex dissociates to give a degraded portion 62 comprising second oligonucleotide sequence 52 and linker 53, a degraded portion 63 that comprises the single stranded sequence 56, and fragments 64 of RNA sequence 60. Dissociation of probe 51 from target polydeoxynucleotide 55 results in release of target polynucleotide 55, which may then hybridize with another molecule of probe 51. In this way, the concentration of degraded portions 62, 63, and 64 increases linearly with time and may be detected by the use of any of the label or label systems discussed herein.

An important advantage of signal amplification with the aforementioned probes relative to the use of single stranded RNA probes is that the probes of this invention are much less susceptible to spontaneous hydrolysis. Accordingly, false background signals are substantially reduced providing higher assay sensitivity.

There are numerous methods for following the progress of the aforementioned signal amplification reaction. For example, because the first oligonucleotide sequence hybridizes to the target polynucleotide and is hydrolyzed, internal hybridization can no longer occur. A fluorescent label, for example, can be attached to either the first or second oligonucleotide sequence. When attached to the second oligonucleotide sequence, a quencher will be associated with the first oligonucleotide sequence. When attached to the first oligonucleotide sequence, a quencher may be attached to either the first or second oligonucleotide sequence. Hydrolysis of the first oligonucleotide sequence causes separation of the fluorescer and quencher in either configuration. Alternatively, multiple fluorescers may be attached to the first oligonucleotide sequence. The fluorescers are spaced such that they are self-quenched. Upon hydrolysis of this oligonucleotide sequence, the fluorescence signal is enhanced. Still another method of detection involves two small molecules such as haptens bound to the probe in a manner that they become separated upon hydrolysis of the first oligonucleotide sequence. In this approach, any immunoassay method that is able to distinguish free from bound hapten such as, for example, ELISA, can be used to monitor this process.

As mentioned above, in one embodiment a probe of the invention is associated with a support. One or more probes of the invention may be associated with a support. Such association may be the result of attachment of the probe to the support directly by bond or linking group. The probe may be associated with the support by being bound indirectly such as through the intermediacy of a group such as by binding a ligand to its receptor or by hybridization of complementary polynucleotides.

The support may be a porous or non-porous, suspendable or non-suspendable, water insoluble material. The support can have any one of a number of shapes, such as strip, plate, disk, rod, particle including bead, and the like. The support can be hydrophilic or capable of being rendered hydrophilic and includes inorganic powders such as silica, magnesium sulfate, and alumina; natural polymeric materials, particularly cellulosic materials and materials derived from cellulose, such as fiber containing papers, e.g., filter paper, chromatographic paper, etc.; synthetic or modified naturally occurring polymers, such as nitrocellulose, cellulose acetate, poly(vinyl chloride), polyacrylamide, cross linked dextran, agarose, polyacrylate, polyethylene, polypropylene, poly(4-methylbutene), polystyrene, polymethacrylate, poly(ethylene terephthalate), nylon, poly(vinyl butyrate), etc., either used by themselves or in conjunction with other materials, flat glass whose surface has been chemically activated to support binding or synthesis of polynucleotides, glass available as Bioglass, ceramics, gels, metals, and the like. Natural or synthetic assemblies such as liposomes, phospholipid vesicles, and cells can also be employed. Also included within the scope of the above are immobilized solid surfaces, that is, surfaces upon which one or more individual supports such as particles have been immobilized. The individual supports have one or more probes of the invention bound thereto. Binding of oligonucleotides to a support or surface may be accomplished by well-known techniques, commonly available in the literature. See, for example, A. C. Pease, et al., Proc. Nat. Acad. Sci. USA, 91:5022-5026 (1994).

One embodiment of the present invention relates to a method for determining a plurality of target oligonucleotides bound at separate individually addressable loci. Each locus may be a separate site on a continuous surface that is individually addressable because of its location such as sites on the surface of a glass or plastic plate. Alternatively, each locus may be an element of a discontinuous surface such as an individual dispersible particle, which is individually addressable because each particle bears a different identifying code. The composition of particles or beads employed in this embodiment may be any one of the materials mentioned above for the support. The particles generally have a dimension of about 0.3 to about 1000 micrometers, usually about 10 to about 100 micrometers.

In the method in accordance with this embodiment of the invention, loci suspected of having different target polynucleotides are incubated with a medium comprising a plurality of probes of the present invention. The incubation is preferably carried out under conditions wherein the second oligonucleotide sequence remains hybridized to the first oligonucleotide sequence in the absence of a target polynucleotide. Contact of a probe with a locus in which the first oligonucleotide sequence of the probe and the target polynucleotide at the locus are complementary leads to hybridization and dissociation of the second oligonucleotide sequence from the hybridized region of the probe. Dissociation of the second oligonucleotide sequence at a locus therefore signals the presence or amount of a target polynucleotide at that locus.

The methods and probes of the present invention have application to the area of arrays. In the fields of biological sciences, arrays of oligonucleotide probes, fabricated or deposited on a surface, are used to identify nucleic acid sequences. The arrays generally involve a surface containing different oligonucleotides or nucleic acid sequences individually localized at discrete sites on the surface. Each array may contain any number of sites, usually at least about 10, frequently at least about 50, but arrays of about 100,000 or more oligonucleotides may be employed for some applications.

Many such arrays are commercially available and may be ordered or coded to permit identification of a particular site either spatially or by a detectable code. Arrays containing multiple oligonucleotides have been developed as tools for analyses of genotype and gene expression and may be prepared by synthesizing different oligonucleotides on a solid support or by attaching pre-synthesized oligonucleotides to the support. Various ways may be employed to produce such an array. Such methods are known in the art. See, for example, U.S. Pat. No. 5,744,305 (Fodor, et al.); PCT application WO89/10977; Gamble, et al., WO97/44134; Gamble, et al., WO98/10858; Baldeschwieler, et al., WO95/25116; Brown, et al., U.S. Pat. No. 5,807,522; and the like.

Arrays used in this embodiment of the invention may comprise probes of this invention but will usually comprise an array of oligonucleotides that, when combined with a solution containing multiple target polynucleotides, causes the target polynucleotides to bind specifically to complementary sites in the array. The array is then incubated with a mixture of probes of the present invention that are designed so that their single stranded regions are hybridizable with each of the target polynucleotides at a site adjacent or near to a suspected polymorphism. Strand displacement across the polymorphic site can occur only if there is an exact match with the hybridized region of the first oligonucleotide sequence of the probe. Displacement can be detected by any means such as, for example, detection of a fluorescent label on the probe or by reversal of quenching of a fluorescer/quencher pair which will cause appearance of fluorescence at a site on the array at which a probe has hybridized with a target polynucleotide. Alternatively, a mixture of labeled oligonucleotides complementary to the second oligonucleotide sequences of the probes can be added. Only labeled oligonucleotides that are complementary to displaced second oligonucleotide sequences bind to the array and are detected. The method provides a means of detecting polymorphisms that is largely independent of secondary structure of the target polynucleotide, temperature, sequence length, or sequence composition. Furthermore, the use of lower temperatures for hybridization in the present method permits the use of more temperature-labile labels.

A variation of the above approach involves attaching different probes of the invention to differently labeled particles. The particles are identifiable by a code associated with the particle. One type of code is based on using different amounts of two or more fluorescers for each type of particle. For example, fluorescent latex particles coded in this manner may be employed; such particles are sold by Luminex Corporation, Austin, Tex. The fluorescence associated with a hybridized probe is then measured along with the coding fluorescence for each particle to permit identification of target polynucleotide molecules or polymorphisms.

The above method of capturing target polynucleotides and binding probes of this invention to the target polynucleotides displayed in an array or on particles is particularly useful for detection of single nucleotide polymorphisms. The most sensitive detection of polymorphisms is achieved when the polymorphic site is within the hybridized portion of the probe and the hybridization is carried out at temperatures below the melting temperature of the hybrid formed between the target polynucleotide and the single stranded region of the first oligonucleotide. In order to assure that strand displacement does not occur when there is a single base mismatch, it desirable to provide a means for reversing inaccurate strand displacement events. As previously noted, this is best achieved by assuring close proximity of the displaced second oligonucleotide and first oligonucleotide. For the assay for the single nucleotide polymorphism, the first oligonucleotide may have a match or mismatch for the single nucleotide polymorphism, preferably a match.

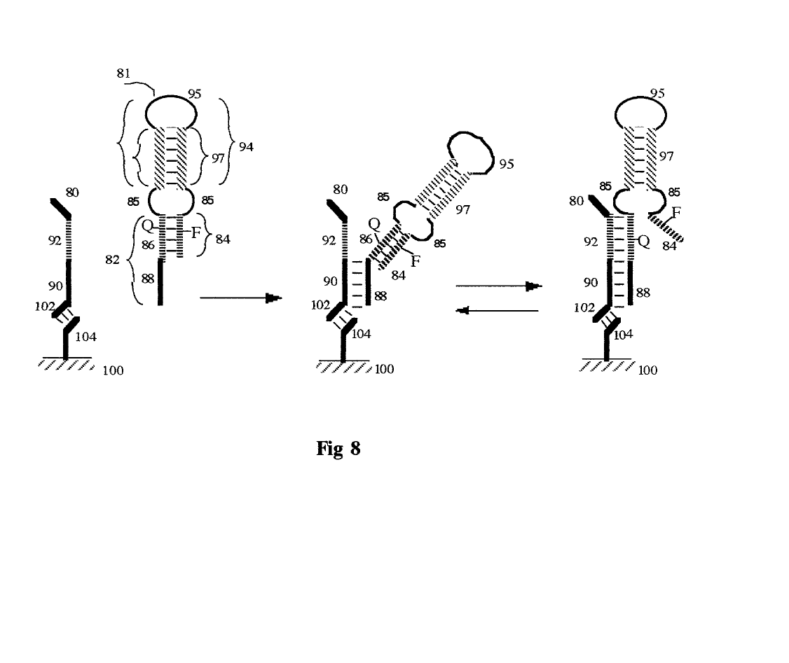

One method for providing this relationship can be understood most readily by reference to FIG. 8. Target polynucleotide 80 is bound to a support 100 by hybridization of portion 102 of the target polynucleotide 80 with an oligonucleotide 104, which is attached to support 100. A probe 81 is used which comprises (1) a first oligonucleotide sequence 82 comprised of a single stranded region 88 complementary to a sequence 90 of the target polynucleotide and a hybridized region 86 complementary to a contiguous sequence 92 of the target polynucleotide; (2) a second oligonucleotide sequence 84 that is complementary and hybridized to the hybridized region 86; and (3) a linker 94 comprised of a double stranded oligonucleotide 97 that is bonded through bivalent connectors 85 to the hybridized region 86 and the second oligonucleotide sequence 84 and that is optionally covalently bonded through a bivalent group 95, wherein 85 and 95 are groups comprising dual functionalities for linking. Sequences comprising the double stranded oligonucleotide 97 are not complementary to target polynucleotide 80 and will usually have at least four nucleotides, more usually at least about 8 nucleotides when covalently bound to each other and will have at least about 10, more frequently more than about 20 nucleotides when not covalently bound.

Assay conditions are used in which the single stranded region 88 is of a length sufficient that its binding to sequence 90 of target polynucleotide 80 is substantially irreversible. Such conditions will be readily apparent to those skilled in the art. Hybridized region 86 can then bind to target sequence 92 by displacement of the second oligonucleotide 84. However, this displacement only occurs if hybridized region 86 is fully complementary to target polynucleotide sequence 92. If a base mismatch is encountered, strand displacement does not proceed further and may reverse direction. Strand displacement can conveniently be monitored when portions 84 and 86 of probe 81 comprise a fluorescer (F) and a quencher (Q) which are positioned to produce a change in the fluorescence of the probe upon dissociation of portions 84 and 86. Usually F and Q are located between the polymorphic site and the linker 94 so that only in the absence of a base mismatch will F and Q be separated and produce a signal. Alternatively, the displaced second nucleotide sequence can be detected by causing a labeled oligonucleotide to bind to it and detecting the amount of the label that has bound.

Another particular embodiment of the present invention involves an array used for the detection of target polynucleotides that differ by a single nucleotide from non-target polynucleotides suspected of being bound to the sites on the array. The nucleotide complementary to the single nucleotide in each of the first oligonucleotide sequences of the present probes is within four nucleotides of the junction of each of the hybridized regions and each of the single stranded regions of the probes. In this approach, as well as those discussed above, each of the single stranded regions and hybridized regions comprise sequences of at least twelve nucleotides.

Another embodiment of the present invention is a method for monitoring the amplification of a polynucleotide. A combination is provided comprising (i) a medium suspected of containing the polynucleotide, (ii) all reagents required for conducting an amplification of the polynucleotide to produce a target polynucleotide, and (iii) a probe as described above. The combination is subjected to conditions for amplifying the polynucleotide to produce the target polynucleotide. Such conditions are dependent upon the type of amplification to be conducted. These conditions are well known in the art and will not be repeated here. Then, the combination is subjected to conditions under which the target polynucleotide, if present, hybridizes to the probe and the hybridized region of the probe dissociates. Such conditions are discussed above. The extent of dissociation of the hybridized region of the probe is detected and is related to the concentration of the target polynucleotide.

The above method may be employed in most amplification reactions such as, for example, PCR, LCR, NASBA, 3SR, SDA, rolling circle amplification and so forth. In each case the progress of the amplification can be followed in real time simply by measuring the signal from the reaction medium.

In a particular embodiment of this aspect of the present invention, by way of example and not limitation, a probe in accordance with the invention can be included in a PCR reaction mixture that includes a polynucleotide to be amplified, nucleotide triphosphates, a polymerase, and appropriate oligonucleotide primers. Both the first and second oligonucleotide sequences of the probe are complementary to sequences in the target amplification product. Conveniently, the probe may have a fluorescer and a quencher and fluorescence is observed only when the probe becomes bound to target amplification product. During the PCR reaction the fluorescence of the solution increases as target amplification product is formed.

As a matter of convenience, predetermined amounts of reagents employed in the present invention can be provided in a kit in packaged combination. A kit can comprise in packaged combination probes as described above for detecting one or more target polynucleotides. The kit may include a reference polynucleotide, which corresponds to a target polynucleotide except for the possible presence of a difference such as a mutation. The kit may include reagents for using the present methods to monitor an amplification of a polynucleotide or for conducting an amplification of target polynucleotide prior to subjecting the target polynucleotide to the methods of the present invention. The kit can include a support having associated therewith an array of oligonucleotides or labeled particles having different oligonucleotides as described above. The kit can include members of a signal producing system and also various buffered media, some of which may contain one or more of the above reagents.

The relative amounts of the various reagents in the kits can be varied widely to provide for concentrations of the reagents that substantially optimize the reactions that need to occur during the present method and to further substantially optimize the sensitivity of the method. Under appropriate circumstances one or more of the reagents in the kit can be provided as a dry powder, usually lyophilized, including excipients, which on dissolution will provide for a reagent solution having the appropriate concentrations for performing a method or assay in accordance with the present invention. Each reagent can be packaged in separate containers or some or all of the reagents can be combined in one container where cross-reactivity and shelf life permit. The kits may also include a written description of a method in accordance with the present invention as described above.

The following examples are intended to illustrate but not limit the invention.

### EXPERIMENTAL

Temperatures are in degrees centigrade (°C.) and parts and percentages are by weight, unless otherwise indicated. The oligonucleotides are obtained from Integrated DNA technologies, Inc. Coralville, Iowa.Materials. Target polynucleotides: Four synthetic ssDNA target polynucleotides tA, tG, tC, and tT and their complementary polynucleotides tAc, tGc, tCc, and tTc are designed arbitrarily and screened to have minimal secondary structure. All polynucleotides differ from one another at one nucleotide in bold type:

| | |
|---|---|
| tA: | |
| 5′-GGTAGGCTTAGGTACCTCAGGATAGAATTTATGTTACCCGCGGTCAATTA3′ | (SEQ ID NO:1) |
| tG: | |
| 5′-GGTAGGCTTGGGTACCTCAGGATAGAATTTATGTTACCCGCGGTCAATTA 3′ | (SEQ ID NO:2) |
| tC: | |
| 5′-GGTAGGCTTCGGTACCTCAGGATAGAATTTATGTTACCCGCGGTCAATTA 3′ | (SEQ ID NO:3) |
| tT: | |
| 5′-GGTAGGCTTTGGTACCTCAGGATAGAATTTATGTTACCCGCGGTCAATTA 3′ | (SEQ ID NO:4) |
| tAc: | |
| 5′-TAATTGACCGCGGGTAACATAAATTCTATCCTGAGGTACCTAAGCCTACC-3′ | (SEQ ID NO:5) |
| tGc: | |
| 5′-TAATTGACCGCGGGTAACATAAATTCTATCCTGAGGTACCCAAGCCTACC-3′ | (SEQ ID NO:6) |
| tCc: | |
| 5′-TAATTGACCGCGGGTAACATAAATTCTATCCTGAGGTACCGAAGCCTACC-3′ | (SEQ ID NO:7) |
| tTc: | |
| 5′-TAATTGACCGCGGGTAACATAAATTCTATCCTGAGGTACCAAAGCCTACC-3′ | (SEQ ID NO:8) |

Four single stranded RNA target polynucleotides trA, trG, trC, and trU have homologous sequences to the DNA target polynucleotides above.

| | |
|---|---|
| trA: | |
| 5′ GGUAGGCUUAGGUACCUCAGGAUAGAAUUUAUGUUACCCGCGGUCAAUUA-3′ | (SEQ ID NO:9) |
| trG: | |
| 5′-GGUAGGCUUGGGUACCUCAGGAUAGAAUUUAUGUUACCCGCGGUCAAUUA-3′ | (SEQ ID NO:10) |
| trC: | |
| 5′-GGUAGGCUUCGGUACCUCAGGAUAGAAUUUAUGUUACCCGCGGUCAAUUA-3′ | (SEQ ID NO:11) |
| trU: | |
| 5′-GGUAGGCUUUGGUACCUCAGGAUAGAAUUUAUGUUACCCGCGGUCAAUUA-3′ | (SEQ ID NO:12) |

Probe Sequences:

All five probes below have a 20 nucleotide (nt) overlap with the target polynucleotides. SDP1, SDP2, and SDP2 have 20 nt single stranded sequences, but the region of hybridization differs. SDP1-l, and SDP1-s have the same structure but different lengths of single stranded sequences, 30 nt and 10 nt, respectively. Q and F represent attached quencher (DABCYL) and fluorescer (tetramethylrhodamine). The underlined sequences are complementary to the target polynucleotide. Bold type indicates the corresponding site of the single nucleotide polymorphism of the target polynucleotides.

SDP1 (Probe 1 for tA) −20 nt single stranded sequence:

| | |
|---|---|
| ₍-AAA-GGTAGG(Q)CTTAGGTACCTCAG-3′ | (SEQ ID NO:13) |
| ⁽-AAA-CCATCC(F)GAATCCATGGAGTCCTATCTTAAATACAATGGGC-5′ | |

SDP1-l (Probe 1 for tA) D 30 nt single stranded sequence:

| | |
|---|---|
| ₍-AAA-GGTAGG(Q)CTTAGGTACCTCAG-3′ | (SEQ ID NO:14) |
| ⁽-AAA-CCATCC(F)GAATCCATGGAGTCCTATCTTAAATACAATGGGCGCCAGTTAAT-5′ | |

SDP1-s (Probe 1 for tA) D 10 nt single stranded sequence:

(SEQ ID NO:15)

₍-AAA-GGTAG(Q)CTTAGGTACCTCAG-3′
⁽-AAA-CCATCC(F)GAATCCATGGAGTCCTATCTTAAA-5′

SDP2 (Probe 2 for tA) D 20 nt single stranded sequence:

| | |
|---|---|
| ₍-AAA-GCTTAG(Q)GTACCTCAGGATAG-3′ | (SEQ ID NO:16) |
| ⁽-AAA-CGAATC(F)CATGGAGTCCTATCTTAAATACAATGGGCGCCAG-5′ | |

SDP3 (Probe 3 for tA)D 20 nt single stranded sequence:

| | |
|---|---|
| (Does not extend to polymorphism) | |
| ₍-AAA-GGTACC(F)TCAGGATAGAATTT-3′ | (SEQ ID NO:17) |
| ⁽-AAA-CCATGG(Q)AGTCCTATCTTAAATACAATGGGCGCCAGTTAAT-5′ | |

SDPC (acyclic control sequence for tA):(SEQ ID NO:18)

AAAAAA(F)AAAAAAAAAAAAAAAAAAAACCATGGAGTCCTATCTTAAAT
ACAATGGGC-5′

### EXAMPLE 1

Detection of single point mutations by strand-displacement using single stranded oligonucleotide targets.

a) Solutions of each of the target oligonucleotides at 10, 1, 0.1, and 0.01 nM are prepared using HYB buffer (500 mM NaCl, 50 mM Sodium Phosphate, 0.1% SDS, pH7 in doubly-distilled deionized water).

b) A 10 ml solution of each of the above 6 probes is prepared in HYB buffer. The solutions are then heated to 95° C. for 1 minute and cooled on ice immediately to ensure proper internal hybridization. target polynucleotide and 10 ml of a probe solution.

c) To each well in a 384-well plate is added 10 ml of one dilution of a target polynucleotide and 10 ml of a probe solution. The mixture is incubated at 37° C. for 30 min, and the change in fluorescence intensity is determined using a Packard fluoroCount [symbol] fluorometer (Packard Instrument Company, Inc. Downers Grove, Ill.).

d) Each probe-target polynucleotide pair is evaluated in duplicate at 4 target polynucleotide concentrations, requiring a total of 8 separate wells. Thus, evaluation of all twelve target polynucleotides with one probe requires 96 wells.

e) Plate 1 is prepared with probes: SDP1, SDP1-l, SDP1-s, and SDPc. Plate 2 is prepared with probes: SDP1, SDP2, SDP3, and SDPc.

f) At each concentration tested, it is found that the fluorescence intensity increases more rapidly with tA and trA than with tG, tT, tC, trG. trT, or trC when using one of the non-control probes SDP1, SDP1-l, SDP1-s, SDP2, and SDP3.

### EXAMPLE 2

Detection of single point mutations by strand-displacement using double stranded polynucleotide targets.

The procedure of Example 1 is followed except that the target polynucleotide solutions are prepared by mixing equimolar amounts of tT and tTc, tA and tAc, tG and tGc, tC and tCc, trA and tAc, trU and tTc, trG and trGc, and trC and trCc so as to provide the same concentrations of target polynucleotides in the solutions. Prior to incubation at 37° C. in step c), the solutions are warmed to 95° C. for 5 min to assure dissociation of the target duplexes. At each concentration tested, it is found that the fluorescence intensity increases more rapidly with tA:tAc and trA:tAc than with tT:tTc, trU:tTc, tG:tGc, trG:trGc, tC:tCc, and trC:trCc.

### EXAMPLE 3

Use of probes to monitor double stranded DNA formed during PCR and single stranded RNA formed during NASBA with point mutation detection.

Materials

Human 5,10-methylenetetrahydrofolate reductase (MTHFR) gene (Genbank accession number NM_{—}005957) has a point mutation C677T (SNP), which is related to an increased risk of cardiovascular disease and neural tube defects. This SNP was previously analyzed using real time PCR (Giesendorf, et al., (1998) Clinical Chemistry, 44(3), 482). The probes of the invention are designed to monitor the PCR amplifications of the MTHFR gene from commercially available genomic DNA. Genomic DNA samples are analyzed for the C677T mutation by conventional PCR followed by Hinf I restriction enzyme digestion and agarose gel electrophoresis according to Frosst, et al., (1995) Nature Genetics 10, 111-113. Three pools of genomic DNA are prepared from C homozygous, T homozygous, and C/T heterozygous sample, respectively. The PCR primers and product amplicon are as follows:

Primers:

(SEQ ID NO:19)

| | |
|---|---|
| 5′-ATGTCGGTGCATGCCTTCAC-3′ | (forward) |

(SEQ ID NO:20)

| | |
|---|---|
| 5′-CTGACCTGAAGCACTTGAAGG-3′ | (reverse) |

Antisense DNA amplicon (112 nucleotides):

5′ATGTCGGTGCATGCCTTCACAAAGCGGAAGAATGTGTCAGCCTCAAAG
AAAAGCTGCGTGATGATGAAATC(G/A)GCTCCCGCAGACACCTTCTCCT
TCAAGTGCTTCAGGTCAG-3′

(SEQ ID NO:21) The SNP site is indicated.

NASBA is performed with the following primers and probes in a PCR thermal cycler without thermal cycling. The probes of the invention are used to monitor antisense RNA amplicon levels in real time and to differentiate point mutations.

NASBA primers:

| | | |
|---|---|---|
| 5′-TAATACGACTCACTATAGGGATGTCGGTGCATGCCTTCAC-3′ | (forward). | (SEQ ID NO:53) |
| 5′-CTGACCTGAAGCACTTGAAGG-3′ | (reverse) | (SEQ ID NO:22) |

The T7 promoter sequence for RNA transcription is underlined. The antisense RNA amplicon is homologous to the DNA amplicon:

| | |
|---|---|
| AUGUCGGUGCAUGCCUUCACAAAGCGGAAGAAUGUGUCAGCCUCAAAGAAAAGCUGCG | (SEQ ID NO:23) |
| UGAUGAUGAAAUC (G/A) GCUCCCGCAGACACCUUCUCCUUCAAGUGCUUCAGGUCAG-3′ | |

The following 3 pairs of probes with total length of 65, 52, and 36 nucleotides, respectively, are used.

SDP1C (probe for the wild type) (SEQ ID NO:24)

(SEQ ID NO:24)

-AAAACGCCC(Q)TCGGCTAAA-5′
-AAATGCGGG(F)AGCCGATTTCATCATCACGCAGCTTTTCTTTGAGGC

3′

SDP1T (probe for the C677T mutant) (SEQ ID NO:25)

(SEQ ID NO:25)

-AAAACGCCC(Q)TCAGCTAAA-5′
-AAATGCGGG(F)AGTCGATTTCATCATCACGCAGCTTTTCTTTGAGGC

3′

SDP2C (probe for the wild type) (SEQ ID NO:26)

(SEQ ID NO:27)

-AAAACGCCC(Q)TCGGCTAAA-5′
-AAATGCGGG (F) AGCCGATTTCATCATCACGCAGC-3′

SDP2T (probe for the C677T mutant) (SEQ ID NO:27)

(SEQ ID NO:27)

-AAAACGCCC (Q) TCAGCTAAA-5′
-AAATGCGGG(F)AGTCGATTTCATCATCACGCAGC-3′

SDP3C (probe for the wild type) (SEQ ID NO:28)

| | |
|---|---|
| -AAACCC(Q)TCGGCT-5′ | (SEQ ID NO:28) |
| -AAAGGG (F) AGCCGATTTCATCATC-3′ | |

SDP3T (probe for the C677T mutant) (SEQ ID NO:29)

| | |
|---|---|
| -AAACCC(Q)TCAGCT-5′ | (SEQ ID NO:29) |
| -AAAGGG(F)AGTCGATTTCATCATC-3′ | |

F=fluorescein, Q=Dabcyl

Polymerase Chain Reaction (PCR)

PCR mixtures are prepared that contain Tag Gold amplification buffer (TaqmanTM PCR core reagent kit, Applied Biosystems, Foster City, Calif.), 4 mmol/L MgCl₂, each of the four nucleotides T, A, G, C (200 mol final quantity), and 20 pmol of each primer in a total volume of 50 ml per tube, 50 ng of genomic DNA, PCR is performed on an ABI 7700 Sequence Detector (Applied Biosystems, Foster City, Calif.). PCR cycling is preceded by 10 min at 95° C. for activation of the Taq Gold DNA polymerase followed by 40 cycles of 30 sec at 95° C., 1 min at 58° C., and 30 sec at 72° C. Following cycling the mixtures are cooled to 37° C. and 15 pmol each of one the probes of the invention and ROX fluorescent dye (Applied Biosystems, Foster City, Calif.) is added to each mixture. The ratios of fluorescein to ROX fluorescence are measured following incubation at 37° C. for 20 minutes. Four replicate PCR amplifications are performed with each of the 6 probes of the invention and each of the 3 genomic DNA sample pools, together with 8 controls with no target polynucleotides, 8 controls with no probe of the invention, and 8 controls with neither target polynucleotide or probe of the invention. Total number of wells is 96.

Each wild type probe produces a high assay response with the normal C homozygous sample pool, an intermediate assay response with the heterozygous sample pool, and a low assay response with the T homozygous sample pool. Conversely, each C677T mutant probe produces a low assay response with the normal C homozygous sample pool, an intermediate assay response with the heterozygous sample pool, and a high assay response with the T homozygous sample pool. Thus, the probes of the invention permit differentiation between the absence of a SNP, heterozygous representation of the SNP and a homozygous representation of the SNP.

When the experiments are repeated using 4 different concentrations of genomic DNA, 50, 10, 2, and 0.4 ng, respectively, the assay response increased linearly with the DNA concentration.

NASBA

5×NASBA buffer contains 200 mM Tris-HCL, pH 8.5, 60 mM MgCl₂, 350 mM KCl, 2.5 mM DTT, 5 mM of each dNTP (Amersham Pharmacia Biotech, Buckinghamshire, England), 10 mM of each ATP, UTP and CTP, 7.5 mM GTP (Amersham Pharmacia), and 2.5 mM ITP (Roche Molecular Biochemicals, Indianapolis, Ind.). The 5×primer mixture contains 75% DMSO and 1 mM each of antisense and sense primers. The enzyme mixture (per reaction) contains 375 mM sorbitol, 2.1 mg BSA, 0.08 Units (U) RNase H, 32 U T7 RNA polymerase, and 6.6 U AMV-reverse transcriptase. All enzymes are available from Amersham Pharmacia, except AMV-reverse transcriptase, which is provided by Seigakaju.

A premixture for a number of reactions is prepared. Each reaction contains 6 ml of sterile water, 4 ml of 5×NASBA buffer and 4 ml of 5×primer mix. The premixture contains 4 ml of water, 1 ml of 20 pmole/ml probe of this invention and 1 ml of 20 pmole/ml solution of ROX [5-(and -6)-carboxy-X-rhodamine, Molecular Probes, Eugene, Oreg.]. The premixture is divided into portions of 14 ml in microtubes. Then 1 ml of purified RNA from a patient is added. The reaction mixtures are incubated at 65° C. for 5 min and, after cooling to 41° C. for 5 min, 5 ml of enzyme mixture is added. The microtubes are then transferred to a ABI Prism 7700 Sequence Detector. Development of fluorescence is followed in a closed tube for 90 min at 41° C. All readings taken are relative to the fluorescence of a reference fluorophore (ROX). The value of fluorescent threshold (Ft) is the time it takes for fluorescence signal to accumulate to certain threshold (100 RFU).

Four replicate NASBA amplifications are performed with each of the 6 probes of the invention and each of the 3 RNA sample pools, together with 8 controls with no target polynucleotides, 8 controls with no probe of the invention, and 8 controls with neither target polynucleotide or probe of the invention. Total number of wells is 96.

All three RNA pools contain the point mutations that are the same as the genomic DNA SNP's. Each wild type probe produces a high assay response (low Ft value) with the normal C homozygous sample pool, an intermediate assay response (intermediate Ft value) with the heterozygous sample pool, and a low assay response (high Ft value) with the T homozygous sample pool. Conversely, each C677T mutant probe produces a low assay response with the normal C homozygous sample pool, an intermediate assay response with the heterozygous sample pool, and a high assay response with the T homozygous sample pool. Thus, the probes of the invention permit differentiation between the wild type, and single point mutant, and 1-1 mixture of wide type and mutant of RNA.

For the wells where probes and RNA targets are perfectly matched different inputs of RNA targets (10, 100, 1000, 10000, 100000, and 1000000 molecules) are found to have different Ft values. The fluorescence signal increases monotonically but nonlinearly with increasing number of molecules. This indicates that strand displacement probes can be used to monitor real time NASBA.

### EXAMPLE 4

Use of probes of the invention having a polypeptide linker where the linker is the label and is a b-galactosidase enzyme donor.

Probes of the invention are used in which an enzyme donor (ED) links two probe sequences, P1 and P2 or P1 and P3 where the members of each pair have different lengths. The sequence pairs, P1:P2 and P1:P3 exist as duplexes with a single stranded region which remains unhybridized. With no target polynucleotide present the probe is cyclic and ED is unable to complement an enzyme acceptor (EA) to produce active enzyme. When target polynucleotide is present, it hybridizes to the unhybridized single stranded region, which initiates displacement of the shorter probe sequence with ring opening. The ED linker of the probe of the invention is then no longer constrained and complements with EA yielding an active enzyme which catalyses hydrolysis of a fluorogenic substrate. Detection of the fluorescent signal indicates ring opening and the presence of a sequence that is complementary to the longer probesequence.

Target polynucleotides:

(SEQ ID NO:30)

| | |
|---|---|
| T1: | 5′-CTTTGGCCACGTGCGCATTCGCTTAGCTAGCCT-3′ |

(SEQ ID NO:31)

| | |
|---|---|
| T1a: | 5′-CTTTGACCACGTGCGCATTCGCTTAGCTAGCCT-3′ |

(SEQ ID NO:32)

| | |
|---|---|
| T1c: | 5′-CTTTGCCCACGTGCGCATTCGCTTAGCTAGCCT-3′ |

(SEQ ID NO:33)

| | |
|---|---|
| T1t: | 5′-CTTTGTCCACGTGCGCATTCGCTTAGCTAGCCT-3′ |

(SEQ ID NO:34)

| | |
|---|---|
| T2: | 3′-GAAACCGGTGCACGCGTAAGCGAATCGATCGGA-5′ |

(SEQ ID NO:35)

| | |
|---|---|
| T2a: | 3′-GAAACCGGAGCACGCGTAAGCGAATCGATCGGA-5′ |

(SEQ ID NO:36)

| | |
|---|---|
| T2c: | 3′-GAAACCGGCGCACGCGTAAGCGAATCGATCGGA-5′ |

(SEQ ID NO:37)

| | |
|---|---|
| T2g: | 3′-GAAACCGGGGCACGCGTAAGCGAATCGATCGGA-5′ |

Probes of the invention have the following sequences linked by ED, namely, P1-ED-P2 and P1-ED-P3, P1:

(SEQ ID NO:38)

| | |
|---|---|
| P1: | 3′-HS-GAAACCGGTGCACGCGTAAG-5′. |

(SEQ ID NO:39)

| | |
|---|---|
| P2: | 5′-HS-CTTTGGCCACG-3′ |

(SEQ ID NO:40)

| | |
|---|---|
| P3: | 5′-HS-CTTTGGCCACGTGCGCATTCGCTTAGCTAGCCT-3′ |

The peptide linker is the synthetic enzyme donor (ED) described in U.S. Pat. No. 4,708,929 as a 43 amino acid b-galactosidase enzyme donor, ED3A, with the exception that the C-terminal amino acid of ED3A is replaced by cysteine thereby providing a cysteine residue at each end of the linker. The â-galactosidase enzyme acceptor (EA) is the cloned 621 amino acid sequence M15 described in the aforesaid patent.

Probes EDPl (P1-ED-P2) and EDP2 (P1-ED-P3) are prepared from the probe sequences which are obtained from BioSource International (Foster City, Calif.) as their bis-disulfides. For preparation of EDP1 the bis-disulfides of P1 and P2 are first hybridized to each other by incubating equimolar amounts in sodium phosphate buffer (100 mM, pH 7.6) at 37° C. for 20 min to permit hybridization. The bis-disulfides of P1 and P3 are similarly caused to hybridize for preparation of EDP2. Buffer containing 0.1M DTT is added to these mixtures which are then incubated under argon at room temperature for 2 hours. The deprotected oligonucleotide partial duplexes are purified by reversed-phase high performance liquid chromatography (HPLC) and used directly to prepare EDP1 and EDP2.

Preparation of activated ED:

ED is treated with the homo-bimaleimide linker, BMH (Pearce Chemical Co. Rockford, Ill.) in phosphate buffer (100 mM, pH 7.6). The corresponding ED-(maleimide)₂ is purified by reversed-phase HPLC.

Preparation of the ED-oligonucleotide conjugates, EDP1 and EDP2:

Each of the deprotected oligonucleotide partial duplexes solutions is added under argon over several hours to separate solutions of 100 mM ED-(maleimide)₂in sodium phosphate buffer (100 mM, pH 7.6) containing about 20% dimethylformamide. The reaction mixtures are purified by reversed-phase HPLC and the peaks corresponding to the one to one adducts are isolated.

Reagents:

EDCB: (ED Core buffer): 100 nM PIPES, 400 mM NaCl, 10 mM EGTA, 0.005% Tween, 10 mM Mg Acetate, and 14.6 mM NaN3, pH 6.83. 10×EA: 0.18 mg/ml EA diluted in EACB. pH 6.83.

EADB (EA dilution buffer): EA Core buffer with 0.5% Fetal Bovine Serum.

EDDB (ED dilution buffer): 10 mM MES, 200 mM NaCl, 10 mM EGTA, 2 mg/ml BSA fragments, and 14.6 mM NaN3, pH 6.5. 4-MUG Substrate: 40 mg/ml of 4-methylumbelliferone-â-galactoside (Molecular Probes) in EACB.

EDP1 and EDP2 solutions are 1, 0.1, and 0.01 nM in EDDB.

Target DNA solutions T1, T1a, T1c, T1t, T2, T2a, T2c, and T2g are 10000, 1000, 100, and 10 nM in EDDB.

Assays are performed on Packard 384 well flat bottom plate (Packard Instrument Co. DownersGrove, Ill.). To each well are added 10 (l EDP1 or EDP2 and 10 (l of a target solution, and the mixture incubated 30 min at 37° C. 10 (l of EA, and 10 (l of 4-MUG substrate are then added and the strand displacement reaction monitored at 0, 30, 60, and 90 min using a Packard LumiCount (Integrated DNA technologies, Inc. Coralville, Iowa).

EDP1 produces an increased signal over background with T1 relative to all the other target polynucleotides and EDP2 produces an increased signal with T2. The signals increase linearly with concentration of T1 and T2 respectively. Increase in the concentration of the other target polynucleotides does not cause significant increases in the signal.

Materials

Probe along with all modifications, synthetic target oligonucleotides and PCR primer sequences are shown below. All were synthesized by Integrated DNA technologies Inc. Coralville, Iowa 52241. Genomic DNA samples were obtained from Coriell Cell Repositories Camden N.J. 08103. Acetylated Bovine serum albumin catalog No. B8894 (“BSA”) and human placental DNA catalog No.D5037 were obtained from Sigma (Sigma Aldrich Saint Louis, Mo. 63103). 10×SD buffer was made from 2M KCL, 1M Tris pH 8.0 stocks (Ambion Austin Tex. 78744). BHQ1 is referred to as black hole quencher and is available from Integrated DNA Technologies, Coralville, Iowa.

1×SD buffer composition:10 mM tris, 50 nM KCl, 0.1% aBSA, pH 8.0 4 mM MgCl₂

I. PROBES

| | |
|---|---|
| (1) P1 | (SEQ ID:NO 41) |

TCTTCTCCTTCCTTCTC(T-F1) GTTGCCACXGTGGCAACA-BHQ1

| | |
|---|---|
| (2) P2 | (SEQ ID:NO 42) |

ACACCAAAGCA(T-F1) CCGGGXCCCGGA-BHQ1

| | |
|---|---|
| (3) P3 | (SEQ ID:NO 43) |

ACACCAAAGCA(T-F1) CTGGGXCCCAGA-BHQ1

The underlined bases represent complementary sequences. X is a hexaethylene glycol backbone. (T-Fl) and (T-T) represent an internal dT carrying Fluorescein or Tamra.

II. PRIMERS (ESTROGEN GENE DERIVED)

| | |
|---|---|
| Fp1 | |
| CCACGGACCATGACCATGA | (SEQ ID:NO 44) |
| Rp1 | |
| TCTTGAGCTGCGGACGGT | (SEQ ID:NO 45) |

(Fp1 and Rp1 intend forward and reverse primers)

III. TARGETS

| | |
|---|---|
| Wt (wild-type) | (SEQ ID:NO 46) |

CACAGAGGCTGAAGTGGCAACAGAGAAGGAAGGAGAAGA

| | |
|---|---|
| M−5 | (SEQ ID:NO 47) |

CACAGAGGCTGAAGTGGCAACAGAGACGGAAGGAGAAGA

| | |
|---|---|
| M+1 | (SEQ ID:NO 48) |

CACAGAGGCTGAAGTGGCAACCGAGAAGGAAGGAGAAGA

| | |
|---|---|
| M+4 | (SEQ ID:NO 49) |

CACAGAGGCTGAAGTGGCCACAGAGAAGGAAGGAGAAGA

| | |
|---|---|
| M+6 | (SEQ ID:NO 50) |

CACAGAGGCTGAAGTGTCAACAGAGAAGGAAGGAGAAGA

(When the target is bound to the probe, the M+x intends the number of nucleotides from the junction of the stem for the presence of the SNP, with the first nucleotide of the double strand of the probe being 1, while M−x intends the number of nucleotides from the junction, with the first nucleotide of the single strand of the probe being 1. The numbering of the target reflects the numbering of the probe.)

| | |
|---|---|
| ESRTa | (SEQ ID:NO 51) |

CAGTAGGGCCATCCCGGATGCTTTGGTGTGGAGGGTCATGG

| | |
|---|---|
| ESRTb | (SEQ ID:NO 52) |

CAGTAGGGCCATCCCAGATGCTTTGGTGTGGAGGGTCATGG

Melt Curve Protocol:

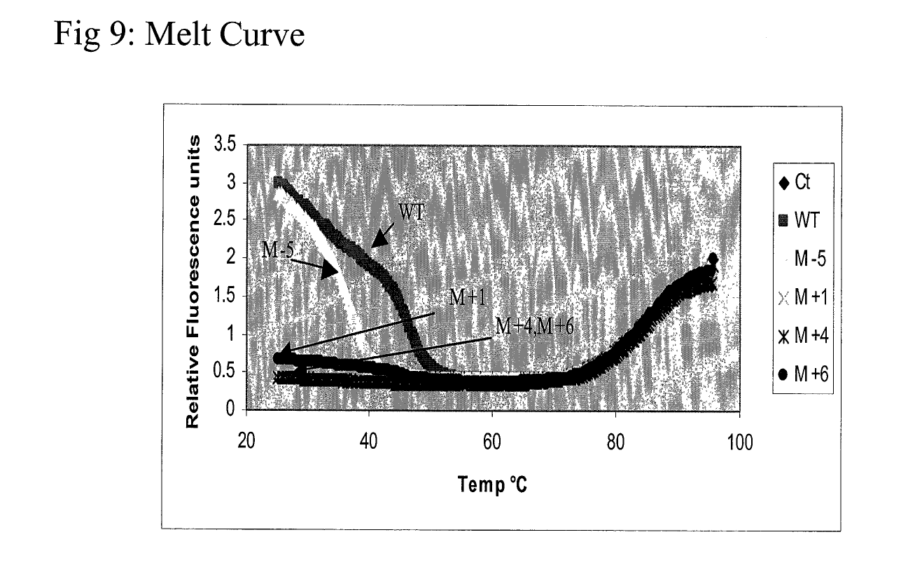

P1 Probe along with various targets was mixed together to a final concentration of 100 nM probe and 300 nM target (Wt, M−5, M+1, M+4, M+6). The reaction was incubated for an hour at room temperature. This was followed by transferring 25 ul of the reaction into 25 ul light cycler capillary tubes. The tubes are spun for a min on a tabletop centrifuge as recommended in package inserts. The melt curves of the Target Probe denaturation from 25° C. to 95° C. were performed in the LightCycler using the Melt Curve Program (Roche Molecular Biochemicals Indianapolis, Ind. 46250-0414). The fluorescence data was plotted against temperature as shown in FIG. 9.

Results

The results (FIG. 9) show a decrease in fluorescence with increasing temperature. The high fluorescence at room temperature with Wt and M−5 targets indicates that these targets have strand displaced and fully hybridized to the probe. This results in the removal of the BHQ quencher from the proximity of the fluorescein molecule. However, as the temperature is increased, the Target/Probe complex denatures, resulting in a stable stem-loop formation bringing the quencher close to the fluorescein molecule. This results in the quenching of the fluorescence exhibited by fluorescein. Increasing the temperature further leads to denaturation of the stem loop structure once again resulting in the separation of the fluorophor/quencher and increased fluorescence.

The very low fluorescence given by M+1, M+4 & M+6 target/probe hybrid signify that even at room temperature the probes cannot displace the stem-loop structure. Hence low fluorescence. The results also signify that mismatches between the probe and the target in the stem region (M+1,M+4,M+6) are not tolerated, as mismatches in the linear portion of the probe are (M−5).

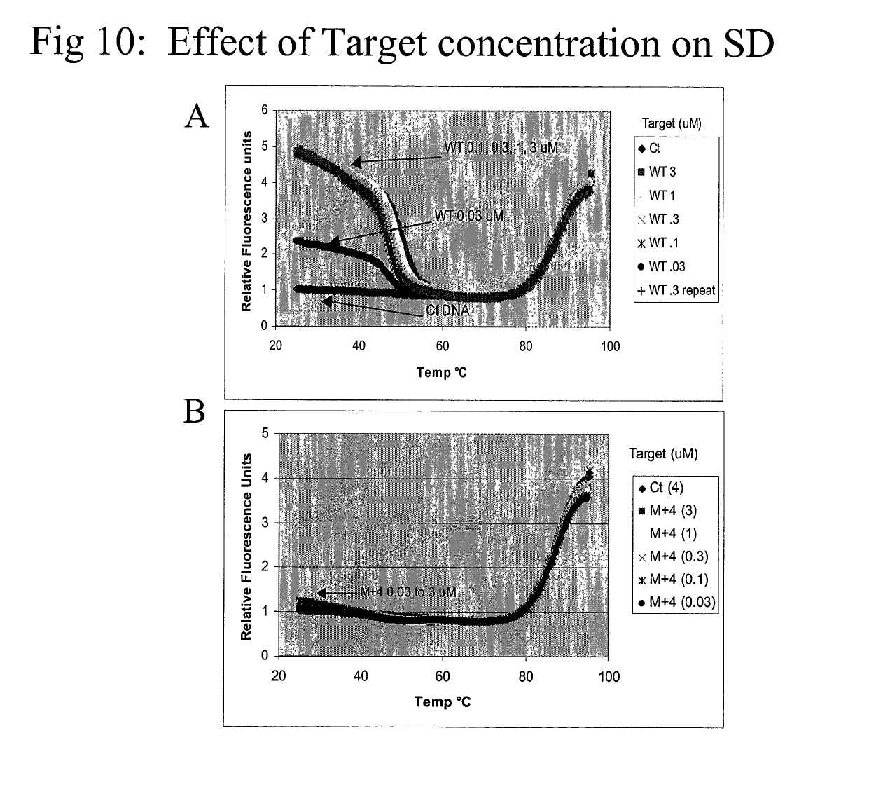

Fluorescence signal is concentration driven as indicated in FIG. 10A. The fluorescence value increases as more specific target (WT) is added to 100 nM probe solution. However the specificity of hybridization is also evident by the fact that the stem-loop probe structure cannot be opened even in the presence of (3 uM) M+4 target (FIG. 10B). Calf-Thymus DNA was also used to indicate the specificity of these probes.

Annealing Kinetics Protocol

The kinetics of probe/target hybridization at room temperature was followed by following the increase in fluorescence from the probe upon target specific opening of the stem-loop probes. Probe P1 87.5 ul was placed in a 50 ul Sterna cuvette in a Perkin Elmer LS 50B fluorimeter. Background fluorescence from the probe in the absence of the target was measured for 5 mins for each read. This is the quenched signal from the probe (stem-loop structure) due to the proximity of the fluorescer and quencher.

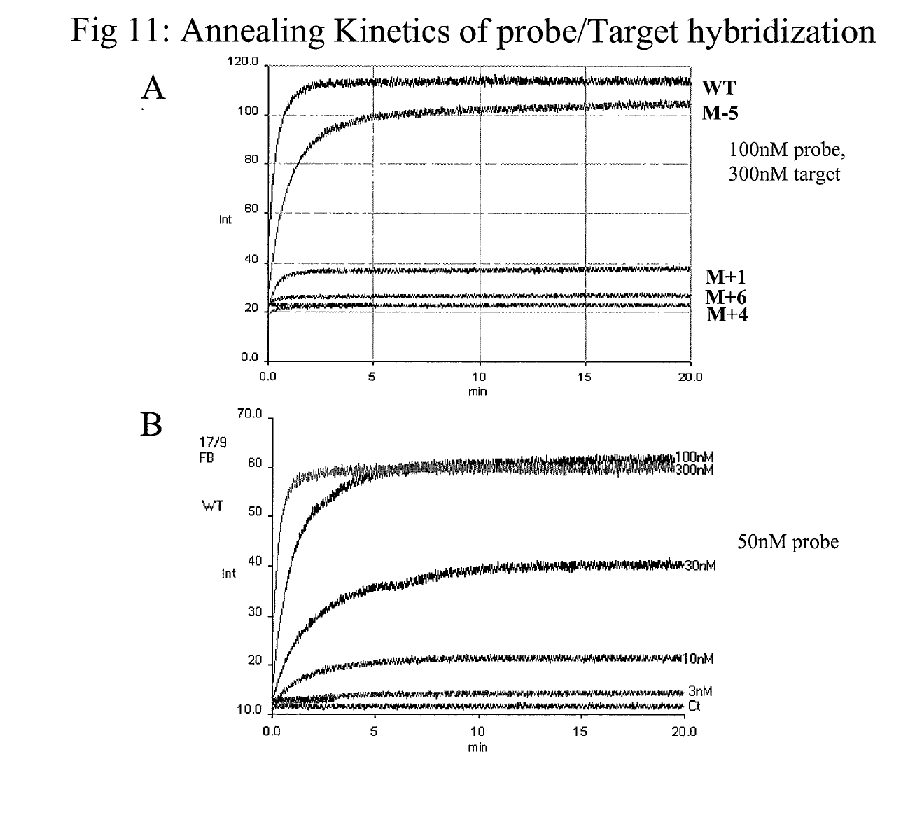

In the same cuvette containing the probe, 12.5 ul of the targets (at various concentrations) were added with the cuvette still inside the fluorimeter. The final probe concentration was at 100 nM or as stated. The lid of the fluorimeter was closed and fluorescence followed with time. Fewer then ten seconds had elapsed between the addition of the target and the start of the data collection. The change in fluorescence signal here is plotted against time and depicted in FIGS. 11A and 11B.

Results

At room temperature, the hybridization kinetics is not only highly specific but also very fast (FIG. 11A) . Mismatch in the single stranded region of the probe allows for partial strand displacement; however, mismatch in the stem region does not allow for strand displacement. In addition the signal obtained is specific (Wt), target concentration driven and stoichiometric (FIG. 11B).

SNP detection Estrogen Receptor codon 10 Protocol

The SD probes were used to show post PCR SNP detection on a SNP in codon 10 of the estrogen receptor gene (GenBank Accession No. M12674). Primers used for carrying out PCR are Fp1 and Rp1. Sequences for probes P2 and P3 are derived from the sequence around codon 10 with the mismatch present in the two alleles placed in the stem region of the probe. Oligonucleotides ESTRa and ESTRb also represent the two allelic sequences around this known SNP and are complementary to the two probes.

Asymmetric PCR was carried out to obtain one of the strands in excess so that probes P2 & P3, which are complementary to it, can bind to this strand without any post PCR cleanup. 100 ng of genomic DNA samples obtained from Coriell was amplified using the following conditions. PCR was carried out in Taq Gold Buffer II with 2.25 uM MgCl₂ in a 75 ul reaction containing: 0.2 uM dNTPs, 2.2 mM MgCl₂, and 6 units of Taq Gold. Primer Fp1 was used at 250 nM while the reverse primer Rpl was at used at 60 nM. Initial Taq Gold activation and DNA denaturation was carried out for 4 mins at 95° C. This was followed by 50 cycles at 95° C. for 18 seconds, 54° C. for 1 min, and 30 seconds at 72° C. A final 5 min at 75° C. step was also included.

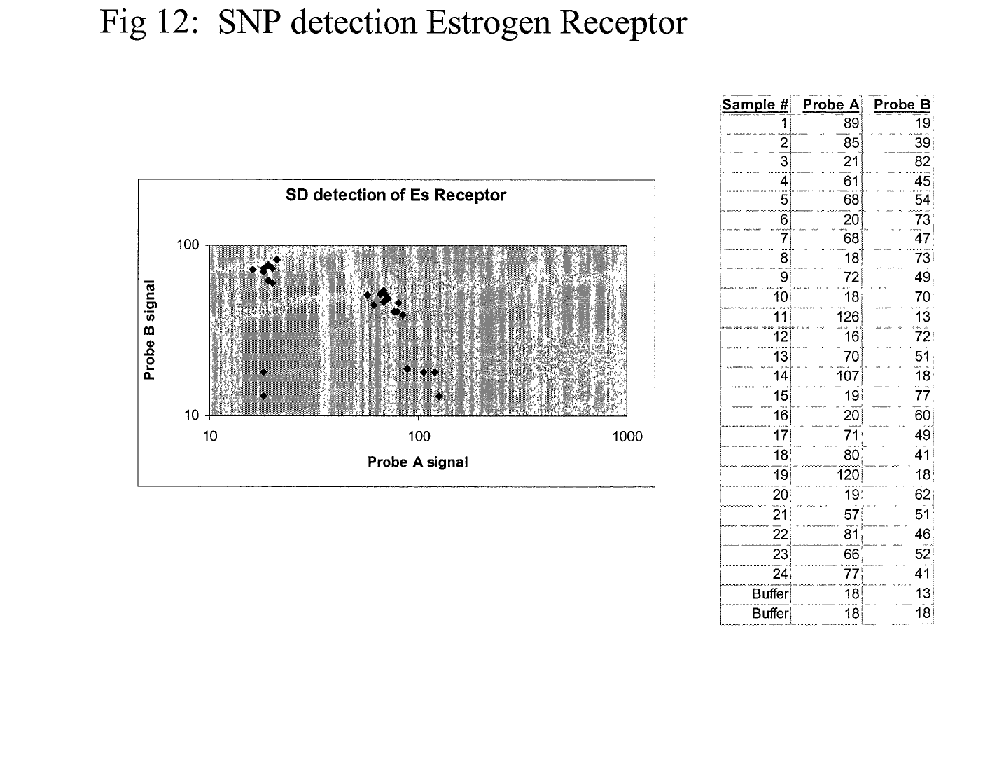

Following PCR, 2 ul of amplified DNA was analyzed on a gel where the asymmetric PCR product migration clearly indicates the genotype of the sample. To determine the SNP in codon 10 of estrogen gene, 25 ul of the PCR amplified sample was mixed with 5 ul of probe 2 or probe 3 in a 384 well black polystyrene plate (Packard). The probes 50 nM final were in SD buffer and the final MgCl₂ concentration was adjusted to 4 mM MgCl₂. The reagents were mixed and the fluorescence read in a fluorescence plate reader (Fluorocount, Packard). The fluorescence was read with excitation at 485 nm and emission at 540 nm. The PMT gain was at one and the PMT voltage was set at 1100 volts. The fluorescence signals from the two probes were plotted as shown in FIG. 12. The raw relative fluorescence units obtained from the amplified sample with probes P2 and P3 are shown in FIG. 12.

Results

The specificity of the two allelic probes for the two targets derived from estrogen codon 10 region is demonstrated in Table 1. The two probes open up with specific targets. Probe P1 gives a high signal with oligo ESTRa and low signal with oligo ESTRb. Similarly, P2 shows high specificity for its complementary target. Even a single base mismatch in the stem region leads to baseline signals at room temperature. These probes were then used to analyze asymmetric PCR products from genomic amplified DNA.

All the amplified DNA samples gave 100% correlation with the result obtained by analyzing the amplified DNA on a 4-20% nondenaturing Novex precast gel (Invitrogen Carlsbad, Calif. 92008). The homozygote wild type, mutant and the hetrozygotic samples were clearly distinguished based on the fluorescence given by the two-allele specific probes.

TABLE 1
Probes RFUs

| Target (nM) | P2 | P3 |
|---|---|---|
| Buffer | 21 | 19 |
| ESRTa (200) | 198 | 18 |
| ESRTb (200) | 29 | 118 |

The above discussion includes certain theories as to mechanisms involved in the present invention. These theories should not be construed to limit the present invention in any way, since it has been demonstrated that the present invention achieves the results described.

The above description and examples fully disclose the invention including preferred embodiments thereof. Modifications of the methods described that are obvious to those of ordinary skill in the art such as molecular biology and related sciences are intended to be within the scope of the following claims.

Although the invention has been described with reference to the above examples, it will be understood that modifications and variations are encompassed within the spirit and scope of the invention. Accordingly, the invention is limited only by the following claims.

What is claimed is:

1. A method for determining a target polynucleotide in a polynucleotide complex mixture, said method comprising:
(a) providing in combination said mixture and a probe comprising,
(1) a first oligonucleotide sequence that is complementary to said target polynucleotide,
(2) a second oligonucleotide sequence that is complementary to and hybridized with a portion of said first oligonucleotide sequence thereby creating a hybridized region comprised of at least five nucleotides of said first oligonucleotide sequence and a single stranded region comprised of at least six nucleotides of said first oligonucleotide sequence, and
(3) a linker connecting said first and second oligonucleotide sequences;
(b) incubating said combination without disassociation of said hybridized region in the absence of binding of target to said probe, and
(c) detecting formation of single stranded said second oligonucleotide sequence as determining said target polynucleotide in said mixture.

2. The method according to claim 1 wherein said linker is other than a polynucleotide.

3. The method according to claim 1 wherein said hybridized region comprises a sequence of at least eight nucleotides.

4. The method according to claim 1 wherein said single stranded region comprises a sequence of at least 15 nucleotides.

5. The method according to claim 1 wherein said probe comprises a molecular energy transfer (MET) pair in molecular energy transfer relationship, whereby molecular energy transfer is inhibited when said second oligonucleotide sequence is single stranded and said detecting is the emission of light from said MET pair.

6. The method according to claim 5, wherein said MET pair is a fluorescer and quencher.

7. The method according to claim 1, wherein said first oligonucleotide sequence is joined to said linker at its 3′ terminus.

8. The method according to claim 1, wherein said target is RNA.

9. The method according to claim 1, wherein said probe has at least one ribonucleotide.

10. The method according to claim 1, wherein said second oligonucleotide sequence is bound at its 5′-terminus to said linker and is blocked from polymerase extension at its 3′ terminus.

11. The method according to claim 1, wherein a plurality of polynucleotides are to be determined, wherein a plurality of said target polynucleotides and a complementary probe for each of said target polynucleotides are included in said combination, said method including the additional step of amplifying said polynucleotides to produce said target polynucleotides.

12. The method according to claim 1, wherein said mixture is suspected of comprising at least 5 target polynucleotides and a probe for each of said target polynucleotides is present and each of said single stranded second oligonucleotide sequences is individually detected.

13. The method according to claim 1, wherein said probe is selected from the sequence SEQ ID: NO 41, 42, and 43.

14. A method for determining a target polynucleotide suspected of containing a single nucleotide polymorphism (snp) in a polynucleotide complex mixture, said method comprising:
(a) providing in combination said mixture and a probe comprising (1) a first oligonucleotide sequence that is complementary to said target polynucleotide, (2) a second oligonucleotide sequence that is complementary to and hybridized with a portion of said first oligonucleotide sequence thereby creating a hybridized region comprised of at least five nucleotides of said first oligonucleotide sequence and a single stranded region comprised of at least six nucleotides of said first oligonucleotide sequence, wherein said hybridized region of said first oligonucleotide is complementary with a portion of said target polynucleotide comprising said snp, and (3) a linker connecting said first and second oligonucleotide sequences;
(b) incubating said combination without disassociation of said hybridized region in the absence of target polynucleotide, and
(c) detecting formation of single stranded said second oligonucleotide sequence as determining the presence or absence of said snp in said target polynucleotide.

15. The method according to claim 14 wherein said linker is other than a polynucleotide.

16. The method according to claim 15, wherein said linker is an aliphatic group.

17. The method according to claim 14 wherein said hybridized region comprises a sequence of at least eight nucleotides.

18. The method according to claim 14 wherein said single stranded region comprises a sequence of at least 15 nucleotides.

19. The method according to claim 14, wherein said first oligonucleotide is joined to said linker at its 3′ terminus.

20. The method according to claim 14 wherein said probe comprises a molecular energy transfer (MET) pair in molecular energy transfer relationship, whereby molecular energy transfer is inhibited when said second oligonucleotide sequence is single stranded and said detecting is the emission of light from said MET pair.

21. The method according to claim 20, wherein said MET pair is a fluorescer and quencher.

22. The method according to claim 14, wherein a plurality of polynucleotides are to be determined, wherein a plurality of said target polynucleotides and a complementary probe for each of said target polynucleotides are included in said combination, said method including the additional step of amplifying said polynucleotides to produce said target polynucleotides.

23. A method for determining a plurality of at least about 5 polynucleotides each suspected of containing a single nucleotide polymorphism (snp) in a polynucleotide complex mixture, said method comprising:
a) amplifying said polynucleotides in said mixture to produce an amplified mixture of single stranded target polynucleotides;
(b) combining said amplified mixture with a probe for each of said target polynucleotides comprising
(1) said first oligonucleotide sequence that is complementary to said target polynucleotide,
(2) a second oligonucleotide sequence that is complementary to and hybridized with a portion of said first oligonucleotide sequence thereby creating a hybridized region comprised of at least five nucleotides of said first oligonucleotide sequence and a single stranded region comprised of at least six nucleotides of said first oligonucleotide sequence, wherein said hybridized region of said first oligonucleotide is complementary with a portion of said target polynucleotide comprising said snp,
(3) a linker connecting said first and second oligonucleotide sequences, and
(4) a label capable of detection as a result of dissociation of said first and second oligonucleotide sequences, under conditions without disassociation of said hybridized portion in the absence of target polynucleotide;
(c) detecting by means of said label formation of single stranded said second oligonucleotide sequence as determining the presence or absence of said snp in said target polynucleotide.

24. The method according to claim 23, wherein said amplification is selected from the group consisting of asymmetric PCR, LCR, NASBA, 3SR, SDA and rolling circle amplification.

25. The method according to claim 23, wherein said label comprises a fluorescer.

26. The method according to claim 23, wherein said label comprises an enzyme donor fragment.

27. The method according to claim 23, wherein said label consists of a MET pair.

28. The method according to claim 27, wherein said MET pair comprises a fluorescer or chemiluminescer.

29. The method according to claim 27, wherein said first oligonucleotide is joined to said linker at its 3′ terminus.

30. A composition of matter comprising an oligonucleotide having a sequence selected from the group consisting of SEQ ID: NO 41, 42, and 43.


SEQUENCE LISTING (53 sequences)

SEQ ID NO: 1 - DNA, 50 bp, Artificial Sequence
  Description of Artificial Sequence Synthetic oligonucleotide
ggtaggcttaggtacctcaggatagaatttatgttacccgcggtcaatta

SEQ ID NO: 2 - DNA, 50 bp, Artificial Sequence
  Description of Artificial Sequence Synthetic oligonucleotide
ggtaggcttgggtacctcaggatagaatttatgttacccgcggtcaatta

SEQ ID NO: 3 - DNA, 50 bp, Artificial Sequence
  Description of Artificial Sequence Synthetic oligonucleotide
ggtaggcttcggtacctcaggatagaatttatgttacccgcggtcaatta

SEQ ID NO: 4 - DNA, 50 bp, Artificial Sequence
  Description of Artificial Sequence Synthetic oligonucleotide
ggtaggctttggtacctcaggatagaatttatgttacccgcggtcaatta

SEQ ID NO: 5 - DNA, 50 bp, Artificial Sequence
  Description of Artificial Sequence Synthetic oligonucleotide
taattgaccgcgggtaacataaattctatcctgaggtacctaagcctacc

SEQ ID NO: 6 - DNA, 50 bp, Artificial Sequence
  Description of Artificial Sequence Synthetic oligonucleotide
taattgaccgcgggtaacataaattctatcctgaggtacccaagcctacc

SEQ ID NO: 7 - DNA, 50 bp, Artificial Sequence
  Description of Artificial Sequence Synthetic oligonucleotide
taattgaccgcgggtaacataaattctatcctgaggtaccgaagcctacc

SEQ ID NO: 8 - DNA, 50 bp, Artificial Sequence
  Description of Artificial Sequence Synthetic oligonucleotide
taattgaccgcgggtaacataaattctatcctgaggtaccaaagcctacc

SEQ ID NO: 9 - RNA, 50 nt, Artificial Sequence
  Description of Artificial Sequence Synthetic oligonucleotide
gguaggcuuagguaccucaggauagaauuuauguuacccgcggucaauua

SEQ ID NO: 10 - RNA, 50 nt, Artificial Sequence
  Description of Artificial Sequence Synthetic oligonucleotide
gguaggcuuggguaccucaggauagaauuuauguuacccgcggucaauua

SEQ ID NO: 11 - RNA, 50 nt, Artificial Sequence
  Description of Artificial Sequence Synthetic oligonucleotide
gguaggcuucgguaccucaggauagaauuuauguuacccgcggucaauua

SEQ ID NO: 12 - RNA, 50 nt, Artificial Sequence
  Description of Artificial Sequence Synthetic oligonucleotide
gguaggcuuugguaccucaggauagaauuuauguuacccgcggucaauua

SEQ ID NO: 13 - DNA, 66 bp, Artificial Sequence
  Description of Artificial Sequence Synthetic probe
cgggtaacataaattctatcctgaggtacctaagcctaccaaaaaaggtaggcttaggtacctcag

SEQ ID NO: 14 - DNA, 76 bp, Artificial Sequence
  Description of Artificial Sequence Synthetic probe
taattgaccgcgggtaacataaattctatcctgaggtacctaagcctaccaaaaaaggtaggcttaggtacctcag

SEQ ID NO: 15 - DNA, 55 bp, Artificial Sequence
  Description of Artificial Sequence Synthetic probe
aaattctatcctgaggtacctaagcctaccaaaaaaggtagcttaggtacctcag

SEQ ID NO: 16 - DNA, 66 bp, Artificial Sequence
  Description of Artificial Sequence Synthetic probe
gaccgcgggtaacataaattctatcctgaggtacctaagcaaaaaagcttaggtacctcaggatag

SEQ ID NO: 17 - DNA, 66 bp, Artificial Sequence
  Description of Artificial Sequence Synthetic probe
taattgaccgcgggtaacataaattctatcctgaggtaccaaaaaaggtacctcaggatagaattt

SEQ ID NO: 18 - DNA, 56 bp, Artificial Sequence
  Description of Artificial Sequence Acyclic control sequence
cgggaaaaaaaaaaaaaaaaaaaaaaaaaaccatggagtcctatcttaaatacaat

SEQ ID NO: 19 - DNA, 20 bp, Artificial Sequence
  Description of Artificial Sequence Primer
atgtcggtgcatgccttcac

SEQ ID NO: 20 - DNA, 21 bp, Artificial Sequence
  Description of Artificial Sequence Primer
ctgacctgaagcacttgaagg

SEQ ID NO: 21 - DNA, 112 bp, Artificial Sequence
  Description of Artificial Sequence Synthetic DNA amplicon
atgtcggtgcatgccttcacaaagcggaagaatgtgtcagcctcaaagaaaagctgcgtgatgatgaaatcrgctcccgcagacaccttctccttcaagtgcttcaggtcag

SEQ ID NO: 22 - DNA, 21 bp, Artificial Sequence
  Description of Artificial Sequence Primer
ctgacctgaagcacttgaagg

SEQ ID NO: 23 - RNA, 112 nt, Artificial Sequence
  Description of Artificial Sequence Synthetic RNA amplicon
augucggugcaugccuucacaaagcggaagaaugugucagccucaaagaaaagcugcgugaugaugaaaucrgcucccgcagacaccuucuccuucaagugcuucaggucag

SEQ ID NO: 24 - DNA, 63 bp, Artificial Sequence
  Description of Artificial Sequence Synthetic probe
aaatcggctcccgcaaaaaaatgcgggagccgatttcatcatcacgcagcttttctttgaggc

SEQ ID NO: 25 - DNA, 63 bp, Artificial Sequence
  Description of Artificial Sequence Synthetic probe
aaatcgactcccgcaaaaaaatgcgggagtcgatttcatcatcacgcagcttttctttgaggc

SEQ ID NO: 26 - DNA, 50 bp, Artificial Sequence
  Description of Artificial Sequence Synthetic probe
aaatcttctcccgcaaaaaaatgcgggagccgatttcatcatcacgcagc

SEQ ID NO: 27 - DNA, 50 bp, Artificial Sequence
  Description of Artificial Sequence Synthetic probe
aaatcgactcccgcaaaaaaatgcgggagtcgatttcatcatcacgcagc

SEQ ID NO: 28 - DNA, 34 bp, Artificial Sequence
  Description of Artificial Sequence Synthetic probe
tcggctcccaaaaaagggagccgatttcatcatc

SEQ ID NO: 29 - DNA, 34 bp, Artificial Sequence
  Description of Artificial Sequence Synthetic probe
tcgactcccaaaaaagggagtcgatttcatcatc

SEQ ID NO: 30 - DNA, 33 bp, Artificial Sequence
  Description of Artificial Sequence Synthetic target polynucleotide
ctttggccacgtgcgcattcgcttagctagcct

SEQ ID NO: 31 - DNA, 33 bp, Artificial Sequence
  Description of Artificial Sequence Synthetic target polynucleotide
ctttgaccacgtgcgcattcgcttagctagcct

SEQ ID NO: 32 - DNA, 33 bp, Artificial Sequence
  Description of Artificial Sequence Synthetic target polynucleotide
ctttgcccacgtgcgcattcgcttagctagcct

SEQ ID NO: 33 - DNA, 33 bp, Artificial Sequence
  Description of Artificial Sequence Synthetic target polynucleotide
ctttgtccacgtgcgcattcgcttagctagcct

SEQ ID NO: 34 - DNA, 33 bp, Artificial Sequence
  Description of Artificial Sequence Synthetic target polynucleotide
aggctagctaagcgaatgcgcacgtggccaaag

SEQ ID NO: 35 - DNA, 33 bp, Artificial Sequence
  Description of Artificial Sequence Synthetic target polynucleotide
aggctagctaagcgaatgcgcacgaggccaaag

SEQ ID NO: 36 - DNA, 33 bp, Artificial Sequence
  Description of Artificial Sequence Synthetic target polynucleotide
aggctagctaagcgaatgcgcacgcggccaaag

SEQ ID NO: 37 - DNA, 33 bp, Artificial Sequence
  Description of Artificial Sequence Synthetic target polynucleotide
aggctagctaagcgaatgcgcacggggccaaag

SEQ ID NO: 38 - DNA, 20 bp, Artificial Sequence
  Description of Artificial Sequence Synthetic probe
gaatgcgcacgtggccaaag

SEQ ID NO: 39 - DNA, 11 bp, Artificial Sequence
  Description of Artificial Sequence Synthetic probe
ctttggccacg

SEQ ID NO: 40 - DNA, 33 bp, Artificial Sequence; identical to SEQ ID NO: 30
  Description of Artificial Sequence Synthetic probe

SEQ ID NO: 41 - DNA, 34 bp, Artificial Sequence
  Description of Artificial Sequence Synthetic probe
tcttctccttccttctcgttgccacgtggcaaca

SEQ ID NO: 42 - DNA, 22 bp, Artificial Sequence
  Description of Artificial Sequence Synthetic probe
acaccaaagcaccgggcccgga

SEQ ID NO: 43 - DNA, 22 bp, Artificial Sequence
  Description of Artificial Sequence Synthetic probe
acaccaaagcactgggcccaga

SEQ ID NO: 44 - DNA, 19 bp, Artificial Sequence
  Description of Artificial Sequence Primer
ccacggaccatgaccatga

SEQ ID NO: 45 - DNA, 18 bp, Artificial Sequence
  Description of Artificial Sequence Primer
tcttgagctgcggacggt

SEQ ID NO: 46 - DNA, 39 bp, Artificial Sequence
  Description of Artificial Sequence Synthetic target sequence
cacagaggctgaagtggcaacagagaaggaaggagaaga

SEQ ID NO: 47 - DNA, 39 bp, Artificial Sequence
  Description of Artificial Sequence Synthetic target sequence
cacagaggctgaagtggcaacagagacggaaggagaaga

SEQ ID NO: 48 - DNA, 39 bp, Artificial Sequence
  Description of Artificial Sequence Synthetic target sequence
cacagaggctgaagtggcaaccgagaaggaaggagaaga

SEQ ID NO: 49 - DNA, 39 bp, Artificial Sequence
  Description of Artificial Sequence Synthetic target sequence
cacagaggctgaagtggccacagagaaggaaggagaaga

SEQ ID NO: 50 - DNA, 39 bp, Artificial Sequence
  Description of Artificial Sequence Synthetic target sequence
cacagaggctgaagtgtcaacagagaaggaaggagaaga

SEQ ID NO: 51 - DNA, 41 bp, Artificial Sequence
  Description of Artificial Sequence Synthetic target sequence
cagtagggccatcccggatgctttggtgtggagggtcatgg

SEQ ID NO: 52 - DNA, 41 bp, Artificial Sequence
  Description of Artificial Sequence Synthetic target sequence
cagtagggccatcccagatgctttggtgtggagggtcatgg

SEQ ID NO: 53 - DNA, 40 bp, Artificial Sequence
  Description of Artificial Sequence Primer
taatacgactcactatagggatgtcggtgcatgccttcac